(19) United States
(12) Patent Application Publication
(10) Pub. No.: US 2016/0310526 A1
(43) Pub. Date: Oct. 27, 2016

(54) Chlorite-Containing Compositions

(71) Applicant: ClearCrescent Technologies, LLC, Annandale, VA (US)

(72) Inventor: Karen C. Swenholt, Annandale, VA (US)

(21) Appl. No.: 15/203,788

(22) Filed: Jul. 6, 2016

Related U.S. Application Data
(63) Continuation-in-part of application No. 15/139,075, filed on Apr. 26, 2016.
Application No. 15/139,075 is a continuation of application No. 12/587,495, filed on Oct. 7, 2009, now Pat. No. 9,320,921.
Application No. 12/587,495 is a continuation-in-part of application No. 14/120,510, filed on May 28, 2014.
Application No. 14/120,510 is a continuation of application No. 13/507,871, filed on Aug. 3, 2012, now abandoned.
Application No. 13/507,871 is a continuation-in-part of application No. 13/573,545, filed on Sep. 21, 2012.
(60) Provisional application No. 62/284,273, filed on Sep. 24, 2015.
(60) Provisional application No. 62/231,420, filed on Jul. 6, 2015.
(60) Provisional application No. 61/274,257, filed on Aug. 15, 2009.
(60) Provisional application No. 61/207,268, filed on Feb. 10, 2009.
(60) Provisional application No. 61/203,375, filed on Dec. 22, 2008.
(60) Provisional application No. 61/663,933, filed on Jun. 25, 2012.
(60) Provisional application No. 61/573,008, filed on Aug. 4, 2011.
(60) Provisional application No. 61/573,198, filed on Sep. 23, 2011.

(51) Int. Cl. A61K 33/20; A61K 45/06; A61K 36/82; A61K 9/00; A61K 36/87; A61K 36/185

(52) U.S. Cl. CPC A61K 33/20; A61K 36/87; A61K 36/185; A61K 36/82; A61K 9/0014; A61K 45/06

(57) ABSTRACT

A composition used to treat a dermatological condition includes a plant-based antifungal and a metal chlorite salt.

### CROSS-REFERENCE TO RELATED APPLICATIONS

This application claims the benefit of priority to U.S. Application No. 62/231,420 filed Jul. 6, 2015 and U.S. Application No. 62/284,273 filed Sep. 24, 2015, each of which is hereby incorporated in its entirety. This application comprises a continuation-in-part of and claims the benefit of priority to U.S. application Ser. No. 15/139,075 filed Apr. 26, 2016, which is a continuation of U.S. application Ser. No. 12/587,495 filed Oct. 7, 2009, which claims the benefit of priority to U.S. Provisional Application No. 61/203,375 filed Dec. 22, 2008, U.S. Provisional Application No. 61/207,268 filed Feb. 10, 2009, and U.S. Provisional Application No. 61/274,257 filed Aug. 15, 2009, each of which is incorporated in its entirety. This application also comprises a continuation-in-part of and claims the benefit of priority to U.S. application Ser. No. 14/120,510 filed May 28, 2014, which is a continuation of U.S. application Ser. No. 13/507,871 filed Aug. 3, 2012, which claims the benefit of priority to U.S. Provisional Application No. 61/663,933 filed Jun. 25, 2012 and U.S. Provisional Application No. 61/573,008 filed Aug. 4, 2011, each of which is hereby incorporated in its entirety. This application also comprises a continuation-in-part of and claims the benefit of priority to U.S. application Ser. No. 13/573,545 filed Sep. 21, 2012, which claims the benefit of priority to U.S. Provisional Application No. 61/573,198 filed Sep. 23, 2011, each of which is hereby incorporated in its entirety.

### BACKGROUND OF THE INVENTION

The present invention relates generally to methods and chlorite containing compositions for the treatment of various disorders and/or diseases of the skin, hair, mucus membranes and fungal and bacterial conditions in humans. The present invention relates generally to methods and compositions for the treatment of dermatological and systemic conditions in humans. It is particularly efficacious for treating infection-induced conditions of the integument and is particularly well adapted for treating fungal infections of the skin, finger and toenails. These methods and compositions are also surprisingly effective against skin disorders such as eczema and psoriasis.

Damage to the integument is from many sources. Many adverse conditions arise from exposure to pathogens of various types. The sources of conditions such as eczema and psoriasis are unclear. Psoriasis may have an inherited component. Without being bound to a particular theory, there exists a need to eradicate pathogens existing in keratinized tissues, especially when such pathogens may trigger a destructive auto immune response.

There appears to be no certain cure for eczema. Treatments currently include steroids and cold packs. Psoriasis symptoms are improved by topical and systemic medications. Both Psoriasis and eczema symptoms tend to recur chronically.

There appears to be no certain cure for onychomycosis. Systemic, topical and laser light therapy are methods currently used in treatment of this condition. A variety of bacteria, dermatophytes, molds and fungus are associated with onychomycosis including but not limited to Trichophyton Rebrum, Trichophyton Mentagrophytes and Candida albicans.

Another method of this invention is a treatment for certain systemic fungal infections such as Candida albicans. MIC studies show synergistic effects against Candida with sodium chlorite treatment and more than additive effects when sodium chlorite is combined with itraconazole against Trichophyton rebrum and Trichophyton mentagrophytes. Candidia albicans and other Candida species, C. parapsilosis, C. tropicalis, C. krusei, and C. lusitaniae also cause an invasive systemic infection having serious consequences. Antifungal resistance in Candida can create refractory infections having very serious consequences. It has been estimated that over 10% of infections acquired in an ICU are caused by Candida. Mortality rates have been estimated at between 25 and 75% depending on how it is attributed to the death.

Other fungal infections have similar treatment with the same group of antifungal agents and similar problems with resistance. These include: Cryptococcus neoformans causing a meningitis, Pneumocystis carinii or Pneumocystis jirovecii species causing a pneumonia, Histoplasma capsulatum causing disseminated disease from a pneumonia, and is particularly dangerous if spread to the CNS, Coccidioides immitis causing pneumonia and sometimes disseminated infection, a few different species of Aspergillis cause a pneumonia, Blastomyces dermatitidis, causing pulmonary infection, Sporothrix schenckii, causing skin infections, Tinea species, causing various skin infections, Paracoccidioides brasiliensis, causing a systemic infection, Zygomycetes. Causing a systemic infection and Trichophyton rubrum, causing skin and nail infections.

Many species causing Microsporidiosis, a gastrointestinal infection, are also treated with a wide variety of antifungals and sometimes result in resistance.

Fungal infections tend to reoccur and are difficult to treat in patients with compromised immune systems, diabetes, steroid treatments, chemotherapy, very old and very young.

### BRIEF SUMMARY OF THE APPLICATION

Without being bound to a particular theory, it appears that pathogens cause diverse dermatological conditions when they invade the keratin layer of skin and/or penetrate layers of keratin in the nail or hoof. There exists a need to eliminate pathogens that have penetrated or partially penetrated skin and nails. Examples of resulting symptoms include horizontal delamination of nails and scaling such as in is evidence with conditions such as psoriasis. After treatment with the methods of the invention described herein, symptoms of horizontal nail delamination and scaling from plaque psoriasis are alleviated. Nails appear unified and scaling is markedly reduced within three days to one month of starting treatment. Systemic effects were not noted when treatments were topically applied according to the methods of this invention.

Though cleaning the surface of the integument is useful, it is theorized that various pathogens can penetrate and survive in the tough top layer of skin and nails which contain keratin. These pathogens can trigger dermatitis in the host which further degrades the skin surface allowing further penetration of pathogens such as but not limited to dermatophytes. There exists a need to deeply clean the keratin-containing top-most layer of skin and keratin based nails and hair. Since diverse pathogens respond to different treatments, there exists a need for a carrier for bioactive compounds that can penetrate keratin, but will not penetrate tissue systemically. Regardless of the origins of adverse skin conditions, there exists a need for effective treatment of their symptoms and methods to cure and prevent recurrence of such conditions.

It is an object of the present invention to provide a method and composition for treating fungal infections.

It is an also an object of the present invention to provide a method and composition for treating internal and systemic infections as well as topical fungal and bacterial infections of the skin, hair, scalp.

It is an also an object of the present invention to provide a method and composition for topically and systemically treating various topical diseases of the skin, hair, scalp.

It is an object of the present invention to provide a method and composition for topically and systemically treating topical diseases of the skin, hair, scalp that fall under the category of dermatitis.

It is another object of the present invention to treating infections generally includes the application of chemical compositions to tissues affected by dermatological disorders.

It is another object of the present invention to treating infections generally includes the application of chemical compositions to the nail and surrounding tissues.

It is another object of the present invention to treating infections generally includes the application of chemical compositions to skin eruptions and rashes, plaques and surrounding tissues.

The present invention method treats infections generally including the application of chemical compositions topically, orally, adsorption across mucus membranes, transdermally or parenterally to the infected and surrounding tissues, or by delivering a composition systemically.

The compositions of the present invention include a salt of chlorite alone or in combination with a bioactive agent. Bioactive agents include but are not limited to a specific antifungal and/or antibiotic agent and/or an oil.

It is an also an object of the present invention to provide a method of treating internal and systemic infections of organs and organ systems.

It is yet another object of the present invention to provide a skin healing composition which aids healing and prevents infection when the skin is broken.

It is another object of this invention to treat necrotizing fasciitis, an infection of superficial fascia, which is a layer of connective tissue below the skin.

It is yet another object of the present invention to provide a skin healing composition which aids the healing of skin disorders caused by hyperkeratosis.

It is yet another object of the present invention to provide a skin healing composition which aids the healing of skin disorders.

It is yet another object of the present invention to provide a cosmetic composition which improves the appearance of skin, nails and hair.

It is still a further object of the present invention to prevent and treat fungal infections in plants and animals.

The present invention prevents and treats fungal infections by using a composition containing a metal salt of chlorite either alone or in combination with known bioactive agents, including but not limited to antifungal compositions.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawing figures depict one or more implementations in accord with the present concepts, by way of example only, not by way of limitations. In the figures, like reference numerals refer to the same or similar elements.

FIGS. 1, 2, 3a, and 3b illustrate treatment of an ingrown toenail.

FIGS. 4-10 illustrate treatment of acne.

FIGS. 11-16 illustrate treatment of psoriasis.

FIG. 17 is a graph illustrating Minimum Inhibitory Concentrations Study (mic) results combining sodium chlorite and itraconazole against Trichophyton rubrum. Results indicate a combined effect.

FIG. 18 is a graph illustrating Minimum Inhibitory Concentrations Study (mic) results combining sodium chlorite and itraconazole against Trichophyton mentagrophytes. Results indicate a more than additive effect.

FIG. 19 is a graph illustrating Minimum Inhibitory Concentrations Study (MIC) results combining sodium chlorite and fluconizole against Candidia albicans. Results indicate a more than additive effect.

FIGS. 20 and 21 are charts illustrating Minimum Inhibitory Concentrations Study (MIC) results combining sodium chlorite and fluconazole against Candida albicans.

FIGS. 22 and 23 illustrate treatment of a dog wound.

FIGS. 24-40 illustrate 6 months of treatment of onychomycosis.

### DETAILED DESCRIPTION

Unless defined otherwise, all technical and scientific terms used herein have the same meaning as commonly understood by one of ordinary skill in the art to which the invention belongs. Although any methods and materials similar or equivalent to those described herein may be used in the practice or testing of the present invention, the preferred methods and materials are described herein. All references cited herein, including published or corresponding U.S. or foreign patent applications, issued U.S. or foreign patents, and any other references, are each incorporated by reference in their entireties, including all data, tables, figures, and text presented in the cited references.

The term “nail conditions,” as used herein, means conditions present anywhere on or under the nail bed including the cuticle that include, but not limited to, nail flaking, nail splitting, tough thickened nails, nail breakage, weathering damage, brittle nails, yellowing nails, paronychia, nail fungus or onychomycosis, bacterial based infection, and cracked nails.

The term “skin conditions,” as used herein, means a condition present anywhere on the skin including, but not limited to acne, and all manifestations associated with acne including but not limited to comedones, enlarged pores and the like. The term “skin conditions,” also includes but is not limited to skin cancer, rashes, blotches; atrophy, dermatitis (including, but not limited to seborrheic dermatitis, erysipelas, erythrasma, forms of eczema including but not limited to Dyshidrosis, eczema, folliculitis, keratoses, hyperkeratosis, melasmas; nodules, nummular dermatitis, precancerous lesions, pruritus, spider veins; senile purpura, warts, wrinkles, sun damaged skin, skin cancer, contact dermatitis, atopic dermatitis, exfoliative dermatitis, perioral dermatitis, psoriasis, and stasis dermatitis), rosacea, impetigo, and other inflammatory skin conditions; minor injury, fissures, deep wounds, necrotizing fasciitis, and the like.

The term “mucosal conditions” as used herein refers to topical conditions on the mucus membranes, particularly those resulting from fungal infections such as vaginal, oralpharyngeal and esophageal infections.

The term “internal conditions” as used herein means non-topical. Internal infections include those limited to a single organ such as the lung in pneumonia or infections that spread to multiple organs or even throughout the entire body as in a septicemia.

The present invention relates to methods and compositions for topically treating infections on the surfaces of the body. These compositions may be used on the scalp, skin, hair, wounds and the like for the purposes of improving the cosmetic condition thereof and inhibiting and treating microbial infections. The compositions of the present invention may also be used to treat fungal infections, particularly those of the nails including onychomycosis.

The compositions of the present invention may also be used to treat animal diseases of nail, hoof, hair, and skin. There exists a need to aid penetration of safely ingestible topical antibiotics and antifungal agents through hair and skin of animals with wounds. There also exists a need to treat deep surface and deep wounds in animals while discouraging animals from licking off medications. The herbal based compositions described herein contain thymol which is an antibiotic and antifungal agent which stops putrefaction and its attendant scent. Though not painful when used topically at concentrations described herein, thymol has a very slight caustic effect on the lips which discourages animals from licking their wounds. The taste is also offensive, thereby eliminating the need for bandages which animals have a tendency to tear off.

In a further embodiment, compositions described herein may be used to treat necrotizing fasciitis, an infection of superficial fascia, which is a layer of connective tissue below the skin. Sodium chlorite enhances penetration of the bioactive thymol. In addition to its antibiotic and antifungal properties, thymol destroys the organized and living ferments, thereby preventing the occurrence of putrefaction.

Sodium chlorite is an antimicrobial agent. It is primarily used for industrial applications such as controlling microbial contamination in industrial cooling systems and towers. It is an oxidizer. Food-processing companies use it for washing fruits and vegetables because it is a fungicide. Since it can destroy natural color matter without attacking the fibers themselves, it is used as a bleaching agent on textiles. When added to an acid, it forms chlorine dioxide, which is used in many municipal water systems to kill microorganisms. Sodium chlorite is currently being used as an ingredient in personal care products such as facial cleansing lotions, acne creams, mouthwash, toothpaste and contact lens preparations for its antimicrobial action and as a preservative in treatments for the eye. Other salts of chlorite have similar uses such as calcium chlorite as a pool shock sanitizer.

In the present invention, alkali and alkaline metal ions are preferred as the salt of chlorite. However, any other cation, whether monovalent, divalent, trivalent, transition metal or other cation may be used such as the ammonium, potassium, magnesium, aluminium, etc. For the purposes of this specification, the chlorite salt is usually referred to by sodium chlorite and the concentrations are weight percentages based on the sodium salt of chlorite. Other chlorite salts may also be substituted for all purposes of the present invention and would have corresponding percentages sufficient to provide the same quantity of chlorite ions.

While measuring sodium chlorite's microbiocidal effectiveness in vitro, creators of previous art determined that the use of chlorite salts in concentrations above 0.75 to 1% would not increase the effectiveness of chlorite salts as an antimycotic. Consequently, only concentrations of less than one percent—sometimes as low as 0.10% have been used as preservatives in compositions and for treatment against fungus infections and other dermatological conditions. Surprisingly, when percentages above 1% were tested in vivo, it was discovered that in addition to its inherent antimycotic effects, sodium chlorite uniquely enhanced penetration of bioactive compounds without irritating skin or nails.

Compositions of the invention are formulated using sodium chlorite at preferred concentrations of 0.2% to 3% (more preferably at 0.75-2.5%), with the most preferred range of 1% to 2%, and at a pH ranging from around 4 to 11, preferably above 6, more preferably around 10. It is preferred that the chlorite be stable and not degrade significantly into chlorine dioxide. A very small amount (less than 30 ppm, preferably less than 10 ppm, more preferably less than 1 ppm of chlorine dioxide) is acceptable prior to use. Sodium chlorite is naturally alkaline when combined with water. Buffers may be used but it is preferable that pH remains above around 3.5 to prevent the chlorite salts from degrading into chlorine dioxide. It is preferable not to include agents such as sodium hypochlorite, or adding buffers that drop the pH below around 3.5. Without therapeutically effective amounts of chlorine dioxide, sodium chlorite alone is highly effective and useful for the purposes of this invention.

The presence of buffering agents such as citric acid or other alpha hydroxy acids bears no relationship to the presence of chlorine dioxide in a sodium chlorite solution. Significantly effective bioactive quantities of chlorine dioxide are only triggered:
At lower pHs (approaching 3 or less) where the chlorite ion (ClO₂_{^{−}}) converts to chlorous acid (HClO₂), which undergoes a complex degradation reaction, from which chlorine dioxide (ClO₂) results.
By the addition of hypochlorite to a mildly acidified chlorite solution. If no acidified hypochlorite (i.e. hypochlorous acid) is present, a solution of sodium chlorite at a pH above 4, although it might contain a citric acid buffer, remains a sodium chlorite solution, NOT chlorine dioxide in solution.
   A chlorite solution, without the addition of an agent, such as sodium hypochlorite at a pH of 6, would not degrade sufficiently rapidly to form a therapeutically effective amount of ClO₂. Except for the addition of sodium hypochlorite, even if a very small quantity of chlorine dioxide is generated at pH 6, it is too small a concentration to have a biostatic or biocidal effect within a realistic time frame.
   It is preferred that sodium chlorite compositions described herein do not contain therapeutic levels of chlorine dioxide; preferably less than 40 ppm chlorine dioxide, more preferably it contains less than 20 PPM chlorine dioxide, and most preferably compositions described herein contain less than 10 PPM chlorine dioxide or less than 1 PPM of chlorine dioxide.

Stabilizers commonly used in the pharmaceutical and cosmetic arts can be used in the compositions of the invention. For example, sodium bicarbonate can be used as a stabilizing agent as can lemon oil and or other citrus oils including grapefruit oil which can also be used as preservatives. When sodium chlorite is used in accord with the instructions in this application, formation of chlorine dioxide was not noted.

In small batches, 4 grams of sodium chlorite dissolved in 150 ml of water with sufficient glycolic acid to drop the pH to 6 is a preferred embodiment. In another embodiment, 2% or 4 grams of sodium chlorite is dissolved in 150 ml of water and no glycolic acid is added. This produces a stable pH 9 or higher solution. Baking soda or other alkaline buffers may be added. Alpha or beta hydroxyl acids may be added. No chlorine dioxide odor is associated with these compositions when they are stored in light-blocking tightly sealed containers.

A preferred embodiment of the present invention includes the use of combinations of chemical compositions containing at least one chlorite salt and at least one other antifungal agent. Examples of other antifungal agents include those of the azole class in effective concentrations, including but not limited to itraconazole, butoconazole, clotrimazole, econazole, miconazole, terconazole, tioconazole, luliconazole, lanoconazole, ketaconazole or any similar antimycotic(s), efinaconazole, particularly those previously known as antifungal agents or those known to inhibit the biosynthesis of ergosterol and cause changes in oxidative and peroxidative enzyme activities leading to an intracellular buildup of toxic concentrations of hydrogen peroxide.

Other antifungal agents that may effectively be used with the compositions of the present invention include but are not limited to terbinafine, tavaborole (Kerydin™), and similar boron small molecule based technology such as but not limited to AN2718, natrifine, amorolfine, amphotericin B, nystatin, natamaycin, naftifine hydrochloride, flucytosine, griseofulvin, potassium iodide, butenafine, ciclopirox, ciloquinol (iodochlorhydroxyquin), haloprogin, tolnaftate, aluminum chloride, potassium permanganate, selenium sulphide, salicylic acid, zinc pyruthione, bromochlorsalicylanilide, methylrosaniline, tribromometacresol, undecylenic acid, polynoxylin, 2-(4-chlorphenoxy)-ethanol, chlorophensesin, ticlatone, sulbentine, ethyl hydroxybenzoate, dimazole, tolciclate, sulphacetamide, urea, benzoic acid and pharmaceutically acceptable salts thereof.

Another method of this invention uses specific organic antifungal and antibiotic agents. Preferably the carrier is an organic oil but other carriers such as but not limited to glycerine and the like are acceptable. The products of the invention contain carrier oils in conjunction with any of thymol, menthol, and camphor, or a combination of two or three of them, in conjunction with essential oils such as but not limited to tea tree oil or menthyl acetate to provide soothing and healing to the skin and nails, particularly of the feet. Of particular value are oils of plant origin. They are useful in treating fungal infections and may be used as prophylactic protection against infections, both bacterial and fungal. For more rapid treatment of fungal infection, products containing plant-based bioactives such as but not limited to thymol and/or menthol in the oils, as taught herein may be given in conjunction with a composition containing sodium chlorite. The invention could be practiced with using a kit having at least two components, on containing sodium chlorite solution and one containing an oxygenating oil and such as thymol with or without menthol. In another version of this invention, Menthol and/or thymol can be used together or alone in combination with an essential oil such as but not limited to tea tree oil and a carrier oil such as but not limited to olive oil. An example of preferred carrier oil is grapeseed oil. An example of a preferred essential oil is menthyl acetate derived from spearmint. Beeswax or other thickeners may be added. An anti-oxidant may be added to create a stable ointment. Glycerin or mineral oil or propylene glycol can be substituted for the carrier oil. Camphor may also be added to the compositions As an example, (A) sodium chlorite solution and (B) the carrier with a phenol and/or an essential oil could then be provided as a two part (A) (B) kit.

Preferred compositions of the invention contain 0.5 to 6% thymol and/or menthol and or menthyl acetate in a carrier oil. Most preferred compositions contain 2.0-3.80% thymol and/or menthol and or menthyl acetate in a carrier oil. The most useful oils for this purpose are plant derived oils. Several oils are used in place of olive oil. Plant and vegetable products include flax oil or linseed oil, safflower oil, hemp, evening primrose, safflower, chia, perilla, and grape seed oils/extracts, sunflower oil. However, sunflower oil gives off a rancid smell when oxidized. Other oils that may be used include walnut candle nut, soybean, corn, wheat germ, canola, sesame, cotton seed oil, rice bran, beechnut, sweet almond, avocado, theobroma, jojoba and castor oil. Some of these oils may give off a disagreeable odor. Most preferred are oils that readily release peroxides when oxidized in the presence of the chlorite. Additionally, other products such as herbal honey, may be used in place of the oils.

Specific antifungal agents that are derived from organic and herbal sources with antifungal properties that may be used in the methods of the invention include but are not limited to eucalyptus crystals, lactic acid crystals, salicylic acid crystals and camphor crystals, thymol and menthol crystals and menthyl acetate. Additionally, oils derived from thymol or menthol or menthyl acetate may be deemed to be used in accord with the methods of the invention. Other beneficial oils, tinctures or crystals (where applicable) may be derived from the following non-limiting list Barberry, bitter almond, cedarwood, Rice bran, cinnamon, citronella, clove, coriander, eucalyptus, frankincense, Garlic, goldenseal, helicrysum, lavender, mustard, myrrh, Neem, Niaouli oregano, parsley, patchouli, Pau D'Arco, pennyroyal, peppermint, red thyme, oil of thyme, oil of horsemint, and oil of Carum Ajowan, De Candolle (Ptychotis Ajowan), a plant from the East Indies, ravensara, rue, sage and tansy, Ides oil, Ribes Nigrum (black currant seed oil) may be used in the method of this invention. Additionally Oils or tinctures derived from California Bay (Umbellularia cahfornica) Beebalm (Monarda didyma) Burdock (Arctium Lappa), especially the root thereof, Western Red Cedar (Thuja plicata) Sweet Root (Osmorhiza occidentalis), especially the root thereof, White Sage (Salvia apiana) California Soap Plant (Chlorogalum pomeridianum) Pacific Wax Myrtle (Myrica cahfornica) California Mugwort (Artemisia vulgaris) Labrador Tea (Ledum glandulosum), Oregon Grape, especially the root thereof in all its forms, (Mahonia ssp.) (Mahonia aquifolia) (Mahonia nervosa). (Hydrastis canadensis), Oxeye Daisy (Chrysanthemum leucanthemum) may also be used in this invention. Essential oils, tinctures or crystals (where applicable) derived from plants containing D-LIMONENE are preferred including lemons, oranges, grapefruit, caraway, dill, bergamot, peppermint, spearmint and their derivatives therefrom such as menthyl acetate, a derivation from peppermint. Ingredients such as manuka oil, colloid silver and diatomaceous earth may be used in this invention. Such organically derived oils and ingredients may also be used in combination with the compositions exemplified herein to augment antifungal action or adjust fragrance.

Several oils are used in place of grapeseed oil or olive oil. Plant and vegetable extracts include flax oil or linseed oil, safflower oil, hemp, evening primrose, safflower, chia, perilla, and grape seed oils/extracts, sunflower oil. However, sunflower oil gives off a rancid smell when oxidized. Other oils that may be used include walnut candle nut, soybean, corn, wheat germ, canola, sesame, cotton seed oil, rice bran, beechnut, sweet almond, avocado, theobroma, jojoba and castor oil. Some of these oils may give off a disagreeable odor. Most preferred are oils that readily release peroxides when oxidized in the presence of the chlorite. Additionally, other products such as herbal honey, may be used in place of the oils if they are capable of producing peroxide upon interaction with the sodium chlorite.

Thymol is a known antifungal agent and antibiotic. It also stops putrefaction, even after it has commenced and removes associated odors. Thymol can be used in combination with olive oil or in place of olive oil in a suitable base in accord with the teachings herein. A non-limiting list of agents similar to thymol may be used in place of thymol or in addition to thymol for beneficial effects include but are not limited to menthol, menthyl acetate, tea tree oil, eucalyptus oil, camphor, and phenol.

A preferred non-limiting example of a composition containing oils that is applied to infected fungal nails and surfaces of the body includes 3.66 g menthol and 3.66 g thymol with 3.66 g menthyl acetate in a base containing 66 ml of grapeseed oil, 33 ml of jojoba oil and 0.25 ounces of beeswax heated to melting point as a thickener.

A glycerin base or the like instead of an organic oil is an effective alternative, though not preferred.

The herbal components may be packaged with the 2% sodium chlorite in one container or two or more containers in a kit to enhance efficacy and stability of components.

Ideally, the bioactive compositions are applied simultaneously with the 2% sodium chlorite solution. A specific antibiotic or antifungal agent may be pre-formulated with the herbal composition or added from another container simultaneously. In the use of a kit, order of application may be varied. It is preferred that sodium chlorite containing compositions are first applied to compromised tissue, hair or nails, followed by the herbal composition. For example, to improve appearance of fungal nails, first a sodium chlorite is applied by dropper to each nail. An additional drop under the nail is useful with a heavily infected great toe. Next the herbal compositions and/or additional specific pharmaceutical antifungals or antibiotics may be applied covering the entire compromised area.

Preferably, the combined ingredients may be briefly rubbed in to the skin and or nail. An alternative preferred method is co-extrusion from a two barrelled container. The sodium chlorite solution may be added to the herbal component in a ratio of 2-3 drops of sodium chlorite to ½ —dime sized portion of the herbal composition and mixed directly on compromised tissue or mixed in hand and applied to the affected area. Customary therapeutically effective concentrations of bioactive agents for topical use are preferred, though with thymol, menthol, and menthyl acetate, percentages as high as 3.8% offer enhanced effectiveness in improving appearance of fungal nails against onychomycosis without causing irritation to periungal tissue. The above composition may be combined with pharmaceutical antifungal agents to improve nail appearance and efficacy. The above composition may be combined to aid skin penetration of target antibiotics against pervasive deep wounds. The herbal composition is also useful as an aid to penetrating and loosening and or removing tartar on teeth in the mouth and dislodging tonsil stones through penetration of tissue.

Another preferred method of the present invention also includes the use of benzoyl peroxide and combinations of chemical compositions containing at least one chlorite salt, in any concentration, preferably above 0.75%, preferably around 2%. This may be used in combination with at least one specific antifungal agent such as as terbinafine.

Depending on the use, formulations for treating the body may have concentrations of chlorite ions as low as about 0.001% and as high as about 10%. It should be noted that chlorite ions are many times less toxic than chlorine dioxide to animal cells (Svecevicius et al, Environ Sci Pollut Res Int. 2005 September; 12(5):302-5, Karrow et al, Drug Chem Toxicol. 2001 August; 24(3):239-58) and that maintaining it at a higher pH will allow for a higher concentrations to be used. Concentrations of up to 0.5% in internal fluids have been shown to be acceptable for internal use. Higher dosages are likely to be acceptable also. External use allows for considerably higher concentrations to be used, and even higher concentrations are acceptable for short-term contact. The upper limit topically is the tolerance to skin irritation, which will vary with the area of skin being treated and the individual. Concentrations of 100 ppm have been shown to be non-toxic to human cells. Ingram et al, Free Radic Res. 2004 July; 38(7):739-50. Likewise when used as a disinfectant, higher concentrations are acceptable.

The processes of the present invention may be used in conjunction with one another not only as a method of treating onychomycosis and other dermatological conditions, and also to control symptoms, to prevent onychomycosis and other dermatological conditions and to maintain the health of the nails after diminishing and controlling the symptoms of those conditions.

The processes and compositions of the present invention may be used in conjunction with one as a method of treating or preventing internal conditions, particularly those caused by fungal infections. As serious fungal infections are frequently hospital acquired or result in particularly susceptible people (immunocompromised, undergoing chemotherapy, transplantation, diabetic, those with circulation problems (PAD, phlebitis, and other similar conditions), burns and other traumatic injury, the very young and very old, etc.). These individuals may be treated prophylactically.

The compositions of the present invention may also be used as disinfectants, particularly for medical equipment that is reused or used for extended periods of time. For example, respiratory equipment, facemasks and lines are prone to be contaminated by various fungi. Even if sterilized, these and other medical devices rapidly become contaminated by aerosols from the environment or other patients in a hospital setting. Likewise, sterile and sanitary manufacturing facilities have need for disinfectants, particularly directed towards fungi. These include pharmaceutical and medical diagnostic and device manufacturing, food processing, cosmetic manufacturing, fermentation, microbial and biotechnology production and the packaging facilities for each.

Compositions of the present invention may be used in a number of other items and for uses other than treatment of infection. Prophylactic and sanitary purposes are most common such as toothpaste, mouthwash and other oral hygiene, douches, swabs, soaks, wound dressings, socks, toe socks and the like and portions thereof.

While this specification is directed towards human use, animal or veterinary uses are equally applicable.

In a previous patent application Ser. No. 12/587,495, the contents of which are incorporated by reference, sodium chlorite is taught to be used at concentrations above 1%, preferably around 2% acts as a penetration enhancing agent for the nail and skin. It further teaches that benzoyl peroxide combined with more than 1%; preferably 2% sodium chlorite forms an effective antifungal that is especially powerful against the dermatophytes that cause athlete's foot and onychomycosis. Additionally, these compositions are especially effective when combined with topical antifungal and antibiotic agents.

Conventional antifungal agents are generally fungistatic. However, when chlorite salts are used alone or in conjunction with antifungal agents against certain fungi including but not limited to Candida albicans, the effect is noted to be fungicidal.

In the present invention, sodium chlorite is pharmacologically stable at pH levels that do not create chlorine dioxide. US Publication No. 2007/0104798, incorporated herein by reference, notes that combinations of peroxides and sodium chlorite are synergistically effective against microorganisms at pH levels, are pharmacologically stable and do not create chlorine dioxide.

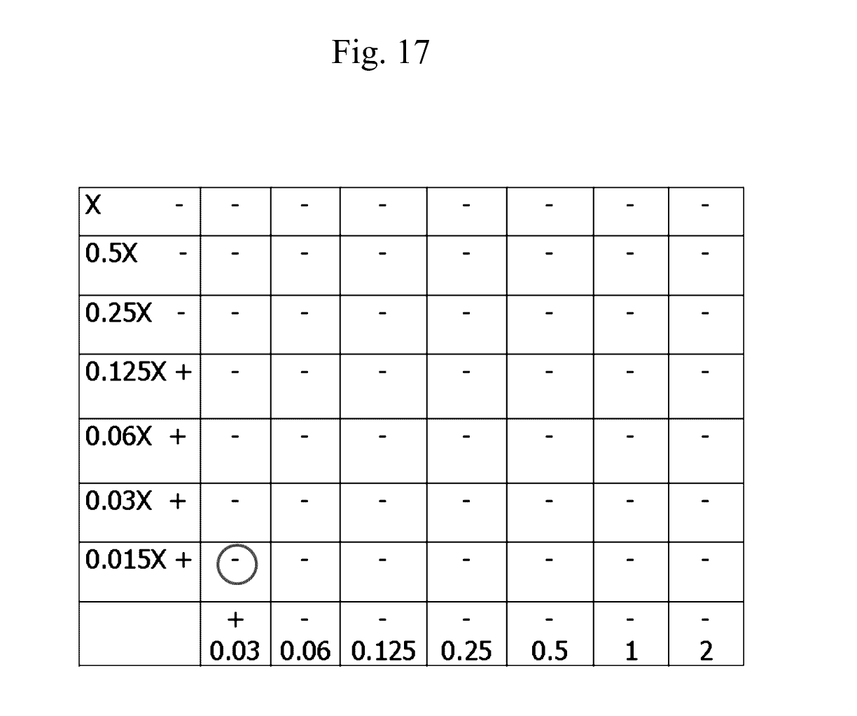

As shown in the examples, in vitro data was generated and the minimum inhibitory concentrations (MIC) tests were determined in broth culture using ATCC MYA 4438. The results are shown in FIG. 17. Itraconazole is the azole used in the study. On the Y-axis are dilutions of the sodium chlorite where undiluted X contains 6.4 micrograms/ml. On the X-axis are dilutions of itraconazole. Fungal Growth is noted by a + and lack of growth by a −.

One can also see that the two compounds combine without antagonism in their antifungal activity. This alone is significant since sodium chlorite is a strong oxidizing agent and quite reactive to a number of organic compounds. When used together, their effect is combined to yield growth inhibition as noted by the data point circled. At those concentrations fungal growth would have occurred if only one compound were present. Therefore, the present invention provides an improved antifungal effect over either compound alone.

The experiments were repeated with a different strain, Trichophyton mentagrophytes ATCC MYA 4439, shown in FIG. 17. Again a significant combined antifungal activity was shown to be superior to either compound alone. For ATCC MYA 4439, the amount of Itraconazole required to inhibit the fungus was decreased at least 4-fold from 0.125 ug/ml to <0.03 ug/ml.

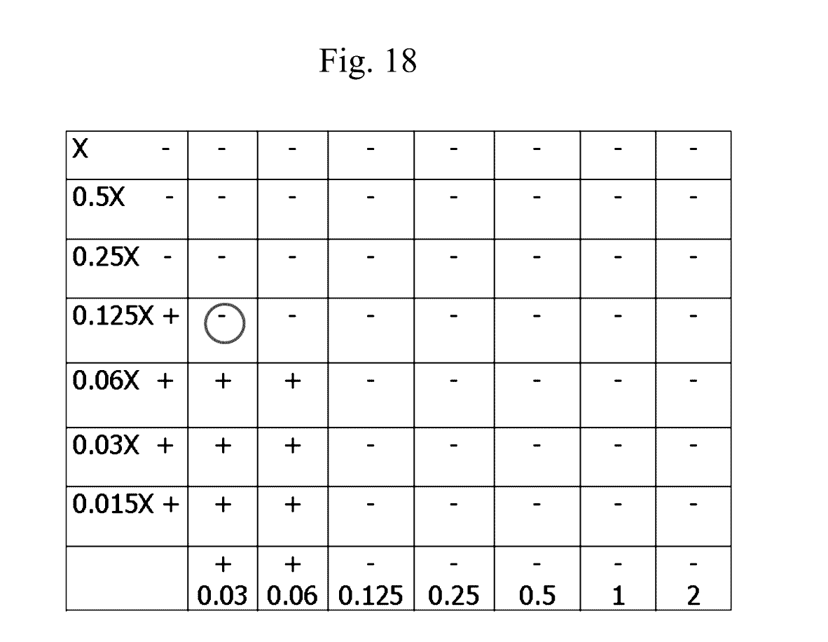

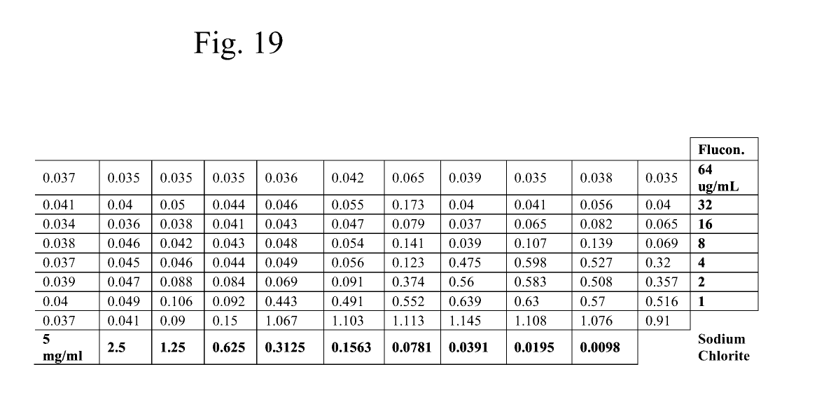

Referring to FIG. 18, the experiments were repeated with the strain Trichophyton rubrum. For T. rubrum, ATCC MYA 4438, Itraconazole was decreased at least 2-fold by dropping from a required amount of 0.06 ug/ml to <0.03 ug/ml.

Azole class antifungals, such as clotrimazole, resolve Athlete's foot within 3 days to one week when they are combined with penetrating sodium chlorite above 0.1%, preferably around 2%. Itching and irritation are soothed within minutes of initial application. Note the in vitro tests featured in FIG. 18 that show a fourfold decrease in the MIC of itraconazole when used with sodium chlorite against Trichophyton mentagrophytes. At present imidazoles take at least two weeks of twice daily treatment for successful treatment of athlete's foot. This in vivo data is confirmed by the in vivo MIC tests against Trichophyton mentagrophytes graphed in FIG. 18. Preliminary testing confirms that up to around 2% sodium chlorite is tolerated in mucous membranes. According to preliminary data, sodium chlorite at concentrations between 0.25-2% will likely augment conventional antifungal based treatment of thrush of the mouth and candidiasis of the vagina and tineas by enhancing penetration of active ingredients thereby speeding results and curtailing treatment time.

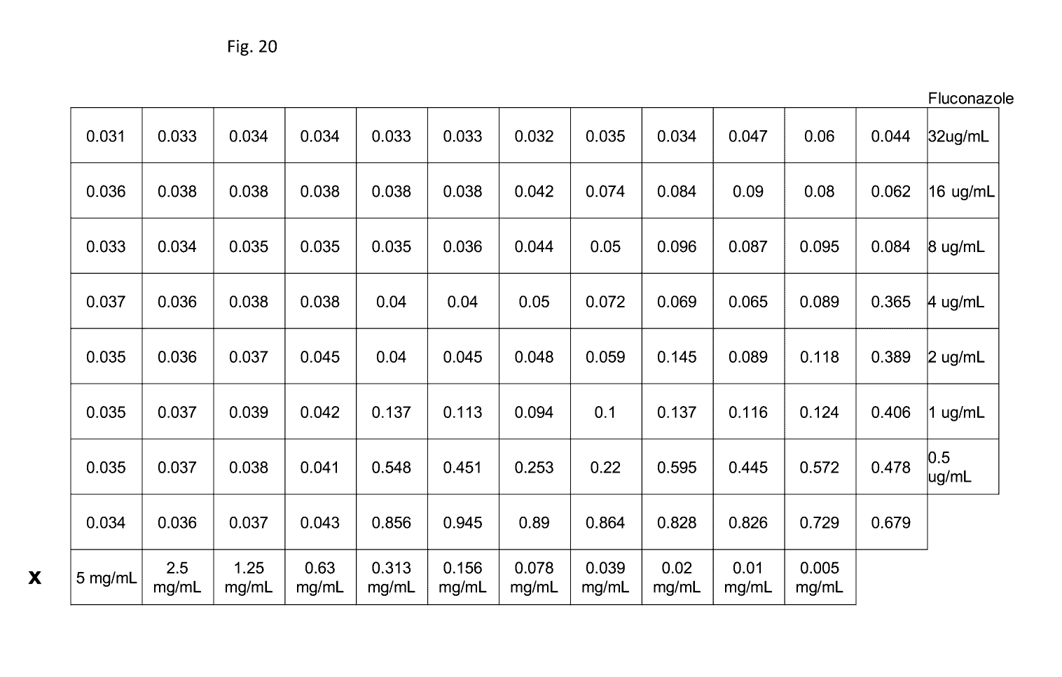

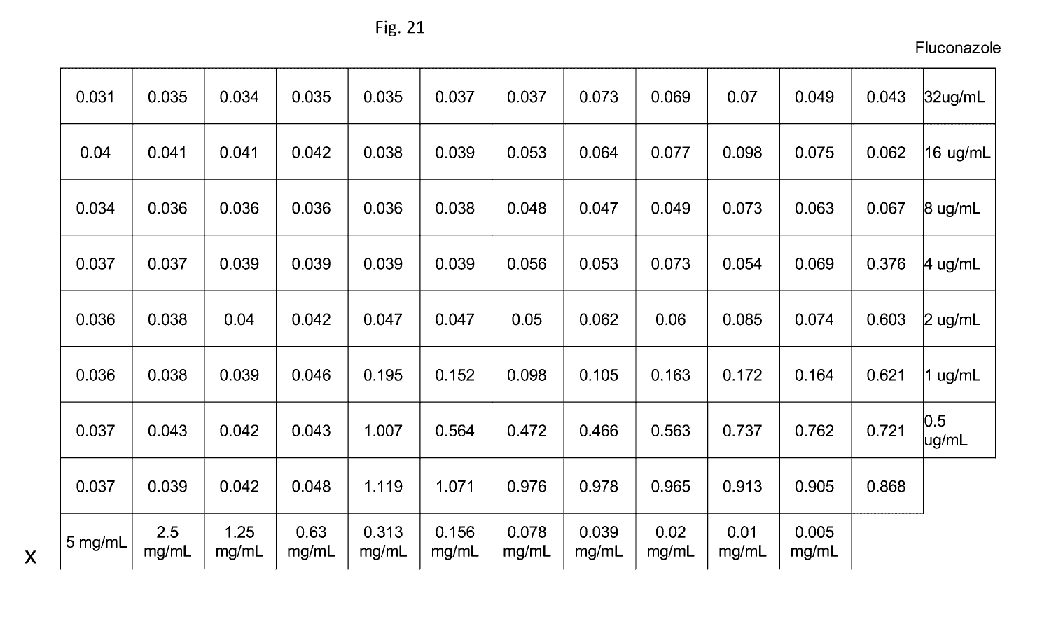

Further, FIGS. 20 and 21 illustrate the results of MIC studies of sodium chlorite and fluconazole against a resistant Candida albicans strain in YPD media after 24 hours. On the Y-axis are dilutions of the sodium chlorite where undiluted X contains 6.4 micrograms/ml. On the X-axis are dilutions of fluconazole. Fungal growth is noted by a + and lack of growth by a −. There is a significant drop in the MICs of both compounds, bringing the concentration of sodium chlorite well below toxic concentrations for human epithelial cells.

These studies indicate that when sodium chlorite is combined with antifungals in the azole group, lower concentrations of sodium chlorite may be used to create an effective antimycotic agent, which is an improvement over previous sodium chlorite containing compositions and previous azole group antifungals. For this purpose, concentrations of sodium chlorite from around 0.015% are effective.

When azoles in concentrations from 0.03% to 15%, preferably 1-3%, most preferably 2% are combined with sodium chlorite in concentrations from around 0.015-3%, positive results can be observed against the dermataphytes responsible for onychomycosis in broth culture and in vivo tests. At higher concentrations from 0.75 to around 3.5%, preferably around 2%, sodium chlorite penetrates the nail and skin without irritation acting as a carrier for the antifungal agent. Sodium chlorite is also effective alone against dermataphytes that cause tinea pedis and onychomycosis in broth culture and in vivo at concentrations ranging from 0.25-4%.

At 0.5%, toxicity against human cells is low and toxicity against Candidas albicans was 100% making ranges around 0.5% the desirable blood concentration with the other ranges provided above acceptable ranges. These concentrations are attainable by oral, injection, infusion and application to mucous membranes. Side by side comparisons with standard antifungal agents gave significantly superior results compared to fluconazole against which it was tested in vitro. Thus, sodium chlorite alone, at pH levels that do not create chlorine dioxide, is effective alone or in combination with antifungals against Candidas albicans.

Broth culture tests also indicate that when combined with azoles against more than one fungus, significant increased antifungal activity has been observed both by enhancing anti-fungal effect of the chlorite salt and simultaneously increasing the effect of the azole (in the case of Trichophyton rubrum, the MIC of itraconazole dropped four-fold; in the case of Trichophyton Mentagrophytes, the MIC of itraconazole dropped two-fold). Further, it has been demonstrated in broth culture tests that when combined with itraconazole against Trichophyton rubrum sodium chlorite's MIC dropped eight-fold. When combined with itraconzaole against Trichophyton metnagrophyte, sodium chlorite's MIC dropped two-fold.

The oil base of the 6.5% tioconazole tested compromised its effectiveness as a treatment option for use on the nail. The oil component of the tested formulation of tioconazole appeared to bloat the nail and discolor it while under treatment. Though tioconazole 6.5% in an oil base was temporarily displeasing aesthetically, it was notably effective against onychomycosis. It is further surmised that safe concentrations of imidazoles above 1-2% such as the 6.5% concentration tested in tioconazole seem to be preferable, though not required for the most effective results. If daily use of such concentrations may not be advisable because of toxicology concerns, higher concentrations of antifungals may be administered at the initial stage of treatment, and then periodically applied on a 1-7 day rotation or a weekly rotation basis. For example, higher concentrations may be used in bursts of 1-7 days followed by treatment at lower concentrations of 1-7 days. Such a regimen pattern could be repeated as necessary until symptoms clear.

Ketaconazole was tested with excellent results when combined with sodium chlorite against fungal infections of the skin and nails.

A preferred method of the aforementioned embodiments combines a cream, lotion or gel base containing 0.5-15%, preferably around 1-2% of a topical antifungal agent or topical antibiotic agent with sodium chlorite, preferably above 1%, more preferably about 2%. This composition may be used to treat dermatological conditions such as, but not limited to nail fungus and bacterial and fungal infections of the skin. A pH between 6-8.8 is preferred. Another most preferred embodiment of the present invention adds 0.005%-5%, preferably about 3% peroxide, preferably but not limited to hydrogen peroxide or benzoyl peroxide with at a preferable ratio of 1 part peroxide containing composition to between 2 parts to 10 parts of the resulting cream, lotion or gel containing sodium chlorite composition. This composition may be used to treat dermatological conditions such as, but not limited to nail fungus and bacterial and fungal infections of the skin such as acne. Also, while corns and bunions are usually not considered infections, a fungal component may be present. Improvement in the size and appearance of corns and bunions in the area of treatment was noticed by test patients during the course of treatment. Therefore, for the purposes of this specification, corns and bunions are considered to be included under skin infections.

Another embodiment of the present invention adds about 0.005%-5%, preferably about 3% peroxide, preferably but not limited to hydrogen peroxide or benzoyl peroxide, to a sodium chlorite composition. Preferred ratios of peroxide to sodium chlorite are 1 part peroxide to between 2 parts to 10 parts of the resulting cream, lotion or gel containing sodium chlorite.

Another preferred method of the aforementioned embodiments is a non-toxic but effective anti-fungal amount of sodium chlorite in oral form such as a capsule or tablet; a parental form such as an injection, intravenously administered solution or infusion to other part of the body, or a transdermal or transmucosal administration. This composition can be used to treat systemic disease or diseases of internal organs. The dosage will vary with the patient and severity of the infection. Suitable dosages are those needed to provide a systemic concentration shown to be effective in vitro without significant toxicity. For example, systemic concentrations from about 0.01 micrograms per milliliter up to about 0.5% may be obtained by administering sufficient sodium (or other metal ion) chlorite to achieve such concentrations. The chlorite salt may be administered alone or in combination with other antifungal agents in one or more compositions.

Another preferred method of the aforementioned embodiments is a non-toxic and effective amount of sodium chlorite in the form of a suppository, cream or gel or solution or an effective concentration of sodium chlorite to form a vaginal rinse. This composition can be used to treat diseases of the reproductive system.

For topical treatments, it is advantageous to include an oil during the treatment. The oil may be intergral to the topical composition containing active ingredients or it may be added afterwards as a protective coating and/or to provide additional effects or to counter the harshness of the active chemicals. Examples include olive oil along with chlorite and an optional antifungal for treating candidia vaginal infections and the like. The treatment may also be used to treat any other topical infections such as ringworm and onychomycosis

Another preferred method of the present invention includes the use of sodium chlorite alone. Sodium chlorite alone is effective against dermataphytes that cause Tinea Pedis (Trichophyton mentagrophytes) and onychomycosis (Trichophyton rebrum) in broth culture at low concentrations. Agar plate tests show that sodium chlorite alone is effective against Candida albicans. At 0.5%, it kills Candida albicans rather than simply inhibiting its growth. It can be concluded that sodium chlorite alone in concentrations significantly below that may be effective against a variety of fungus.

Another advantage to the present invention is that when stable sodium chlorite that does not form chlorine dioxide is used alone and in combinations described herein, the chemical compound(s) are superior against pathogenic fungi than currently recognized antifungal agents alone.

One advantage of the present invention is that it is safer than current conventional antifungal oral medications in that it does not harm the user's liver. Furthermore, the interaction between certain antifungal medications inhibiting the P450 complex and other medications are known problems. The use of sodium chlorite avoids such problems.

Yet another advantage of the present invention is that the preferred combinations are likely to be pharmacologically stable at recommended pH levels for eighteen months. Even with off-gassing of chlorine dioxide, a number of mouthwashes and other oral hygene products are sufficiently shelf stable in dry form. The present invention, using a higher pH and less or no release of chlorine dioxide is even more stable.

Compositions used in the present invention include those in one container and those prepared from multiple containers. The nature of some of the chemicals used can make them react with each other over time resulting in a product, which lacks one-year (or greater) shelf stability. In such a situation, the present invention may be a kit containing two or more containers of chemicals such that chemicals reactive with each other are kept in separate containers. Compounds that are stable together are preferably premixed. The contents of the containers may be mixed immediately prior to use, added simultaneously or sequentially at the target site of infection or may be mixed together ahead of time and briefly stored before use. For example, some components may be mixed in liquid form and stored for example, about two months or so whereas the original components may be shelf stable for over one year. Storage under refrigeration or freezing conditions may extend the acceptable storage time. The final kit may include packaging for the two or more containers and instructions for preparation and use. Individual containers may contain any form of solids, liquids, gels, ointments, creams, etc. At least one of the containers or an additional empty container may be resealable after dispensing its contents after each application.

The final form of one or more components to the composition of the present invention is preferably liquid or spreadable solid (paste, gels, powders, ointments, etc.). A soap product containing some of the components of the composition of the present invention may be formed. This soap may be either in liquid or solid bar form. Components that are incompatible or volatile may be provided separately such as in a dropper bottle. In such a way a person may wash and simultaneously treat infected nails or prophylactically apply some or all of the components to the nails, and if necessary followed by application of one or fewer liquids to the nails. Optionally, one may formulate a treatment bar with a different solidifying agent that resembles a soap bar but without the active soap ingredient. One example would be with the use of stearyl alcohol as the solidifying agent.

In an embodiment, chlorite salts or oxychlor compounds may be applied directly to skin disorders and/or infections of the mucous membranes, and/or hair, and/or infected nails, nail bed and surrounding skin. The sodium chlorite compound may be applied in any form, but it is preferred that the compound is in solution or emulsion form.

In a third embodiment, the method of treating dermatological conditions and disorders according to the present invention includes the use of sodium chlorite, as a penetration enhancer when used with fungicides and other bioactive agents. Sodium chlorite appears to increase the diffusion of existing topical treatments through skin, particularly keratin-containing skin and nail layers and in the case of the nail through to the nail bed. Whether or not any of the agents described herein as penetration enhancers actually increase penetration, they appear to operate well in conjunction with other active compounds in the composition. For the purposes of this application, compositions that provide an added effect to other active ingredients when used in the intended use environment are sometimes referred to as penetration enhancers. Penetration enhancers as used herein are targeted and do not substantially harm the skin.

The various embodiments of the present invention may be used alone or in conjunction with one another.

The invention of this application has been described above both generically and with regard to specific embodiments. Although the invention has been set forth in what is believed to be the preferred embodiments, a wide variety of alternatives known to those of skill in the art can be selected within the generic disclosure. The invention is not otherwise limited, except for the recitation of the claims set forth below.

### EXAMPLES

### Example 1

Compositions were topically applied to various skin and nail conditions. When sodium chlorite is used in an aqueous solution, it consisted of 4 grams of 80% sodium chlorite salts dissolved in 150 ml water. The solution was applied by dropper to affected areas, or combined with a bioactive agent and then applied to the affected area or applied sequentially to the affected area at least once daily, preferably twice daily during treatment period. Sodium chlorite can also be combined in one container with a bioactive such as but not limited to Clotrimazole lotion and maintain its stability.

### Example 1.1

### Eczema

Background: A 21-year old male subject was diagnosed with Dyshidrosis in March of 2012. Symptoms included small blisters on the palms of his hands and the underside of his fingers with scaling. He was treated with Triamcinolone Acetonide 0.1% with little improvement. He abandoned the treatment. Symptoms continued. He occasionally self-treated the condition with over the counter Lamisil. Results of Lamisil treatment included stinging but no significant amelioration of symptoms.

Treatment: For this study, Subject J combined 10% efinaconazole in the form of Jublia and a two percent sodium chlorite and water solution by dropper onto hands in a 1:1 ratio and then distributed the combined composition to the entire affected area.

RESULTS: After one application, flaking of skin ceased. One week after one treatment as described, blisters were healed, leaving slight purple marks where they had been present. The slight purple discolorations faded, resolving to the appearance of normal skin within two weeks. Twenty days later, all symptoms are entirely gone with no relapse.

TABLE 1
Eczema (Dyshidrosis)

| | |
|---|---|
| Location of | Symptoms |
| symptoms | Small blisters oozing yellow serum; scaling |
| Palm, sides | |
| of fingers | |
| Frequency | 10% Efinaconazole + 2% Sodium chlorite |
| of | and water solution |
| treatment | 1:1 ration applied by dropper |
| One | |
| application | |
| Result after | After 10 days |
| one | No Rash, No scaling; slight purple marks |
| treatment | where blisters had been. |
| | After 20 days |
| | Healthy appearing skin. No relapse |

### Example 1.2

### Psoriasis

Background: A 61-year-old female subject suffered from Psoriasis diagnosed while in her late twenties. She has not undergone treatment for her condition since 1996. She reports rash after bathing that turns to white plaques with scaling within days.

Treatment 1: After applying tape to divide her right elbow into two lateral sections, she applied 10% efinaconazole in the form of Jublia alone to the interior lateral section. Next she combined 10% efinaconazole and a two percent sodium chlorite and water solution by dropper in a 1:1 ration in her hands. She then distributed the combination to the entire affected right lateral section of her elbow.

RESULTS: 12 hours later, symptoms on the outside section of her elbow treated with the combination drugs were significantly reduced. Three days later the outside lateral section appeared clear of white scaling, with diminished rash. The side treated with efinaconazole alone had white dry scales and evidence of rash.

Treatment 2: After applying tape to divide her right elbow into two lateral sections, she applied 1% luliconazole in the form of Luzu alone to the interior lateral section. Next she combined 1% luliconazole and a two percent sodium chlorite and water solution by dropper in a 1:1 ration in her hands. She then distributed the combination to the entire affected outside lateral section.

RESULTS: One day later symptoms on the combination drug side were similar to the side treated with Luliconazole alone. White dry scales were perceived though the rash was less apparent on both sides.

After 3 days, red rash had faded to a lighter pink on the side treated with luliconazole and sodium chlorite. No plaques were present. On the side treated with Luliconazole alone, red rash and plaques were present.

Treatment 3: After applying tape to divide her right elbow into two lateral sections, she applied 1% Clotrimazole to the interior lateral section. Next she combined 1% clotrimazole and a two percent sodium chlorite and water solution by dropper in a 1:1 ration in her hands. She then distributed the combination to the entire affected interior lateral section.

RESULTS: Two days later symptoms on the combination drug side were similar to the side treated with Clotrimazole alone. White dry scales were not perceived and the rash was less apparent on both sides.

Surprisingly, subject reports that 92 days after treatments 1 and 2, her symptoms on her elbows are less pronounced. She makes no distinction as to which side has superior long range effects.

TABLE 2
Psoriasis Treatment

| | | |
|---|---|---|
| Treatment 1: | Interior Elbow | Exterior Elbow |
| Location | | |
| Left elbow | | |
| Treatment | 10% efinaconazole | 10% efinaconazole + 2% |
| | | Sodium chlorite and |
| | | water solution |
| Result after 3 days | Rash and white scaling | Rash is discernibly |
| treated twice daily. | present | lighter pink. No scaling |
| | | is present |
| Treatment 2: | Interior Elbow | Exterior Elbow |
| Location | | |
| Left elbow | | |
| Treatment | 1% Luliconazole cream | 1% Luliconazole |
| | | cream + 2% Sodium |
| | | chlorite and water |
| | | solution |
| Result after 3 days | Red Rash and scaling | Rash is lighter pink. |
| treated twice daily. | are present | No plaques present |

TABLE 3
Psoriasis Treatment

| | | |
|---|---|---|
| Experiment 3: | Interior Elbow | Exterior Elbow |
| Location | | |
| Left elbow | | |
| Treatment | 1% Clotrimazole cream | 1% Clotrimazole |
| | | cream + 2% |
| | | Sodium chlorite |
| | | and water solution |
| Result after 3 days | Red Rash present, but | Rash is discernibly |
| treated twice daily. | plaques are not present | lighter pink. No |
| | | plaques present |

### Example 1.3

### Treatment of Psoriasis on Chin

Patient was treated on the chin for three days twice daily. On her right side of the chin, sodium chlorite solution was applied alone. On her left side, sodium chlorite was applied according to Composition 16, Table 12. (The composition used consisted of 2% sodium chlorite mixed in hand with a pre-prepared composition containing 3.66 g thymol, 3.66 g. menthol, 3.66 g. menthyl acetate in 100 ml grapeseed oil w/0.25 ounce melted beeswax). 92 days later, subject reports there has been no recurrence of psoriasis symptoms in the treated area. She reports that both sides of her chin have not shown signs of psoriasis since treatment. Analysis of the photos indicates that previous to treatment the right side of her chin showed more pronounced symptoms. After treatment, skin and scaling on both sides are markedly free of rough skin and scaling. The right side does show 1 raised lesion with appearance similar to acne at 36 days and 3 at 92 days. However, no scaling was reported on either treated area.

Cheeks and nose were left untreated as a control. Symptoms in untreated areas of the face continued to persist throughout 92 days during which cessation of symptoms on the treated chin was photo-documented.

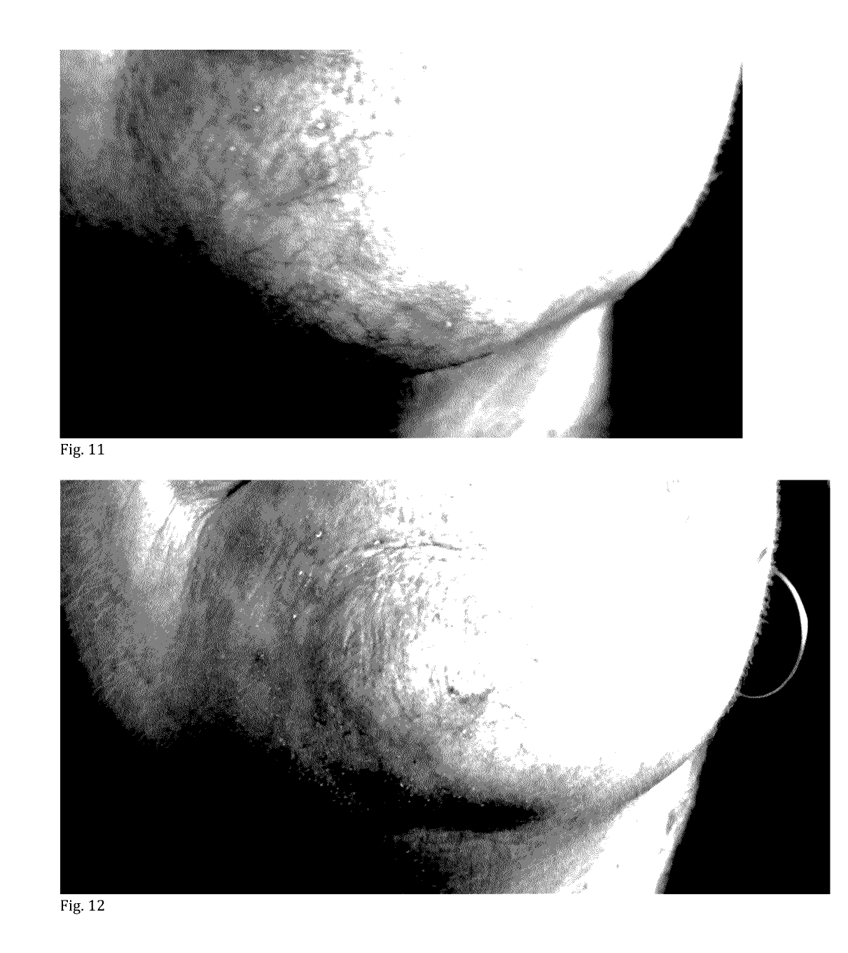

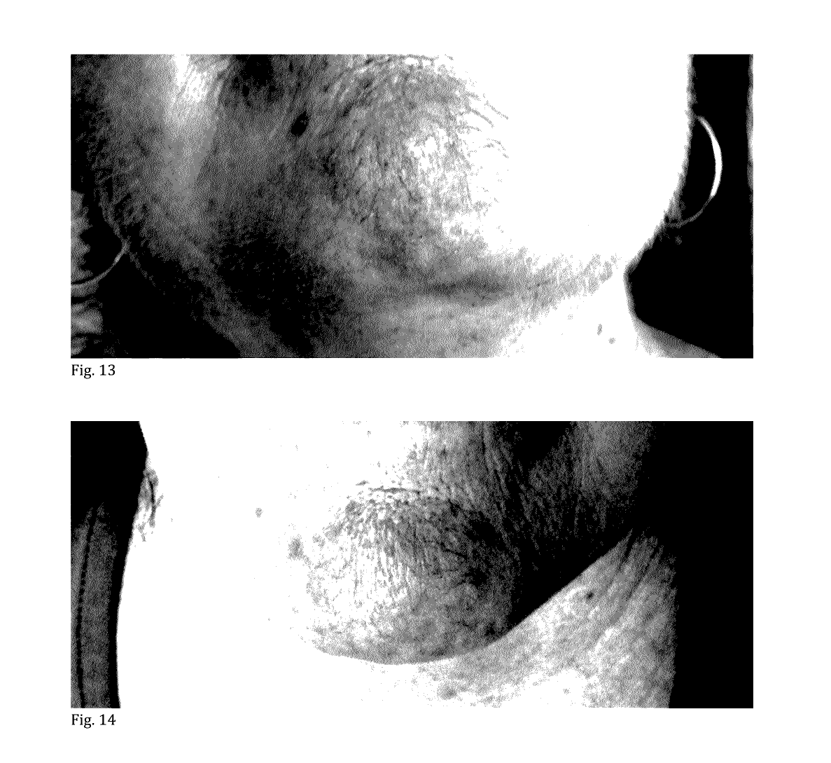

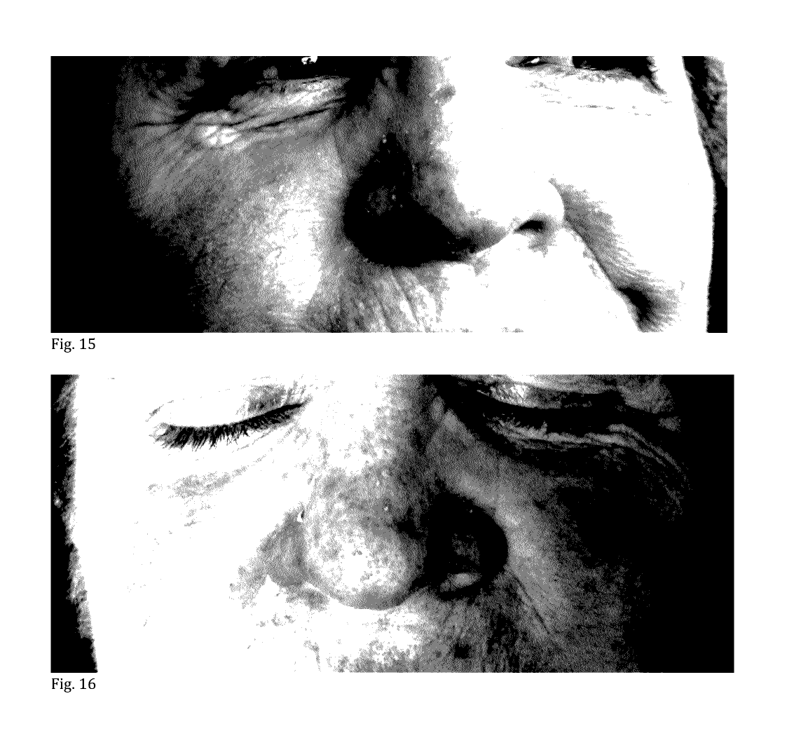

FIGS. 11 and 12 illustrate the patient's chin prior to treatment, and FIG. 15 shows the control areas of the nose and cheeks. FIG. 13 illustrates the patient's chin after treatment twice daily for 3 days according to the following protocol: She applied SCpH10 to her left side of her chin. She applied SC combined with CCT's herbal treatment to her right side of her chin. SC+ a composition containing 3.66 g thymol, 3.66 g menthol, 3.66 g menthyl acetate in 100 ml grapeseed oil with 0.25 ounce beeswax. 36 days later Subject reports that she has not experienced psoriasis symptoms since treatment. FIG. 14 illustrates that, 92 days after initial treatment, no scaling or raised white lesions. Subject reports clear skin. FIG. 16 shows that, 92 days after initial treatment of chin, cheeks and nose still show signs of psoriasis.

Subject self-treated cheeks, nose and forehead with SC+a composition containing 3.66 g thymol, 3.66 g menthol, 3.66 g menthyl acetate in 100 ml grapeseed oil with 0.25 ounce beeswax, taking care to avoid the eye area 3 times by mixing in hand in a 1:2 ratio (sodium chlorite to herbal composition) and then applying compositions to affected areas. She reports clearer skin without pronounced scaling after 3 months.

### Example 1.4

### Hyperkeratosis; Corns

Sodium chlorite in example 3a was present in a composition containing 2% sodium chlorite and 2% Clotrimazole in a cream base.

3a. Subject L had three prominent corns on his toes. Within one week after beginning treatment with Composition S (combined 2% Sc and 2% Clotrimazole), 3 corns of varying intensity began to reduce in size and pain. By day 21, one minor corn was gone, a second almost completely gone, and a third greatly reduced.

3b. Treatment was begun with 2% sodium chlorite and water solution+3% thymol and 3% menthol combined with 100 ml grape seed oil and beeswax to form a balm. As treatment progressed, the reduction of the most prominent corn (which was surmounted by a white hardened pimple atop raised reddened skin) continued to regress rapidly over the next two weeks. The white hardened pimple is gone and the skin is only slightly reddened. Pronounced callusing is reduced. Treatment was abandoned and one corn returned but the (two other corns did not return. Patient whose corn had been reduced by his podiatrist was told the corn would come back. When the pimple began to form again, treatment was applied again. Corn reduced in size and the pimple disappeared. Apparently sodium chlorite and azoles or sodium chlorite and thymol and/or menthol reduce and/or can eliminate symptoms from corns. Sodium chlorite is a known anti-inflammatory. This result surpasses the work of previously used anti-inflammatory agents. Reduction of corns is significant.

### Example 2

### Acne Treatment

TABLE 4
Components of Acne Data Defined

| Antibiotic: | Consistent choice of antibiotic is sold as Neosporin. It | |
|---|---|---|
| | contains Bacitracin 400 units, Neomycin 3.5 mg, and | |
| | Polymyxin B 5000 units. Alternatively, oral antibiotics | |
| | are also acceptable though not preferred. | |
| Benzoyl | “Proactiv Repair Lotion” uses 2.5% Prescription grade | |
| peroxide: | | |
| | “Clean & Clear” uses 10% Prescription grade | |
| | “On the Spot” uses 2.5% Time release | |
| | “Acne Free” uses 10% Time release | |
| Antifungal | A1 | 2% Miconazole cream in Miconazole 7 |
| Agent: | | |
| | A2 | 1% Terbinafine gel in Lamisil AT Gel |
| | A3 | 1% Tolnaflate in Mycocide NS |

| | |
|---|---|
| Compositions: | Sodium chlorite is 2.0% sodium chlorite or four grams |
| | sodium chlorite dissolved in 5 ounces of water. pH |
| | ranges of 4.5-10, preferably around pH 6-7 were used. |
| | All other compounds are pure, as described and are |
| | mixed with water unless otherwise stated. |
| | Liquid was measured by drops from a standard |
| | pipette. |
| | 1 Part of a solid is defined as ribbon 0.5 cm wide and |
| | 0.5 cm long. |
| Treatment: | Subjects washed affected area once a day unless |
| | otherwise noted. All treatment regiments were |
| | repeated at least twice daily. Compositions remained |
| | after application until the next application or bathing. |

Back ground of Acne treatment: Subject T is a 17-year-old boy with very sensitive pale skin had severe acne on his back, forehead, less on the lower face. Subject T saw a Dermatologist. He was prescribed systemic daily antibiotic, 100 grams doxycycline and Prascion cleanser with 0.025 gram Retin A gel. Treatment was not begun because of concerns about Retin A side effects, particularly the prediction that skin might blister, crust and/or get worse before it got better, * and mother's concerns that ingested antibiotics might compromise his natural immunity. The dermatologist told him that he had deep comedones, and odulocystic lesions. He was told that he had a form of deeply based pernicious sub dermal bacterial acne that could only be addressed by ingested antibiotics. The patient explained that he had had a persistent purple toned discoloration on his left cheek for more than a year that troubled him.

### Example 2.1

### Facial and Back Acne

Repeated Studies: To help determine whether good results from SC treatment could be attributed to the episodic waxing and waning characteristic of acne, two separate 4-7 day tests were conducted, followed days later by a 21-29 day study. The tests consisted of recommended twice daily applications of compositions. Not only were good results consistently obtained during the 4-7 day tests, (with the exception of round three which contained a different composition) but comparatively good appearance and reduction of lesions were maintained during the 21-29 day testing period, which was not characteristic of their complexions before treatment.

TABLE 5
Subject T Rounds of Treatment

| | |
|---|---|
| Round 1 | 4 days |
| Interval 1 | 31 days |
| Round 2 | 6 days |
| Interval 2 | 240 days |
| Round 3 | 29 days |

TABLE 6
Subject J Rounds of Treatment

| | |
|---|---|
| Round 1 | none |
| Interval 1 | 31 days |
| Round 2 | 6 days |
| Interval 2 | 240 days |
| Round 3 | 10 hours |

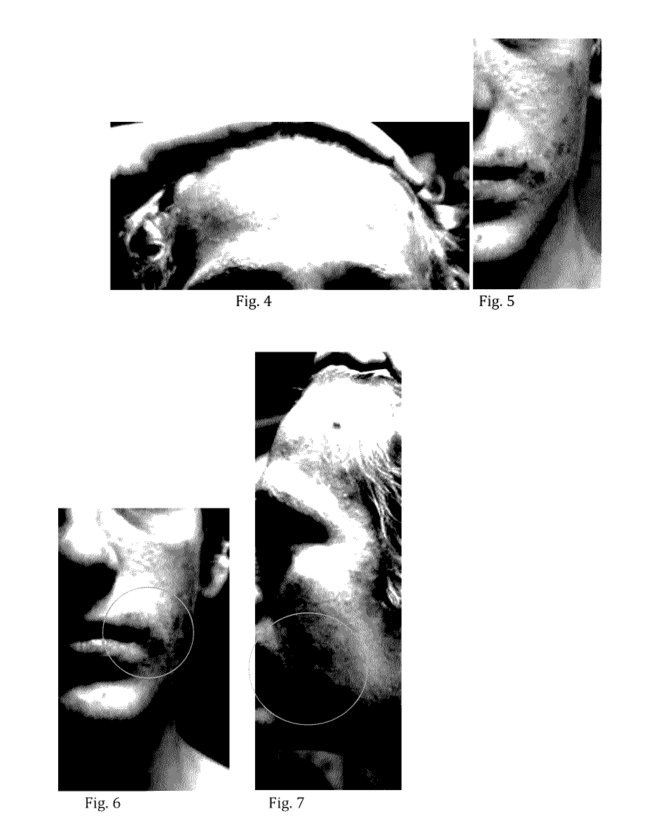

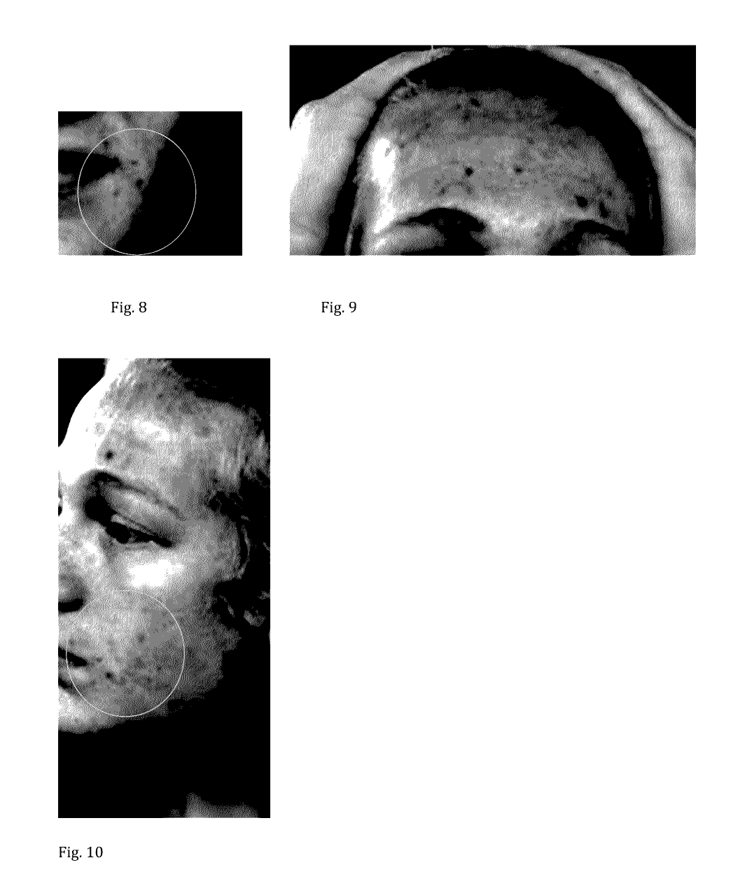

In Rounds 1 and 2, prior to treatment, Subject T regularly used the same benzoyl peroxide acne cleaner by Proactiv as was used in the compositions applied in this study. Therefore, before pictures shown in FIGS. 4 and 5 illustrate results of “control” therapy. After pictures shown in FIGS. 6-10 illustrate the results of the application of preferred embodiments of these compositions. Specifically, FIGS. 4 and 5 illustrate the infected area 40 minutes after the first treatment. FIGS. 8-10 illustrate the infected area after 3.5 days. In FIG. 10, the forehead is smooth.

Before Round 3, Subject T was already using an adequate acne treatment, Clearasil pads and Pronexin. Though comedones had been reduced both in size and frequency of eruption at onset of treatments described here, he still had deeply based sub dermal bacterial acne for which he had been prescribed ingested antibiotics. Thus far, he has never been treated with antibiotics because of his mother's objection to it.

Note that after treatment for 28 days with an unusually mild concentration of 1.33% SC, miconazole, and 2.5% BP, the pernicious sub dermal acne causing lumps and bumps on his forehead are significantly reduced.

1.80% SC liquid was applied to a 1″ tall by 2.5″ wide section of subject's back and observed at 15 minute and 40 minute intervals. There was no sign of redness or report of irritation.

When 1.80% SC was combined with BP, either 2% or 10% redness was reported.

For Round 4, SC concentration for Subject T (sensitive skin) was dropped to 1.33% and only 2% Bp was used throughout.

Round 1: Subject T began this treatment on his back on Dec. 12, 2009. The next day, Dec. 13, 2009 treatment was begun on the face. The first treatments on both areas targeted outbreaks, not the entire surface area of the face and back.

RESULTS (Please see photos): Within 4 days of beginning treatment, all treated skin is markedly clearer. Purplish mark that patient complained about has faded.

Round 2: Treatment was resumed. Product was reapplied for 6 days. Please see table and photos.

Round 3: Treatment was applied for 29 days. Please see table and photos.

### Example 2.2

### Facial and Back Acne

Large Comedone was treated with 2% sodium chlorite solution mixed in palm of hand with equal parts Jublia, then applied to lesion. Pain was reduced after application. Comedone was significantly reduced in size by day 2 but was still tender to the touch. By day 3, mark left by comedone was not easily distinguished by a quick scan of skin.

TABLE 7
Acne/Pore Reduction Concentrations of
Compounds A, B, B1, C1, D, E, G, and H.

| | | | | Micon- | | Clotri- |
|---|---|---|---|---|---|---|
| | Benzoyl | Sodium | Glycolic | azole | Anti- | mazole |
| | Peroxide | Chlorite | 2.5% | 2% | biotic | 2% |
| A | 10% | 2% | | 2 parts | 1 part | |
| B | 2.5% | 1.5% | | 2 parts | 1 part | |
| B1 | 2.5% | 1.5% | | 2 parts | 1 part | |
| C1 | 1.5% | (2p) | 2 part | 2 parts | | |
| | | 1.5% | | | | |
| D | 2.5% | 1.33% | | 2 parts | | |
| E | 2.5% | — | | 2 parts | | |
| G | 2.5% | 2% | | 2 parts | | |
| H | 2% | 1.88% | | 2 parts | | |

Combinations were mixed in hand, and then applied simultaneously except in the case of undiluted sodium chlorite, wherein sodium chlorite was applied after other compounds were mixed and applied undiluted.

TABLE 8
Irritation Key

| | |
|---|---|
| I1a | No redness |
| I1b | no sensation |
| I2a | Moderate to limited redness |
| I2b | Tingling |
| I3a | Moderate to extreme redness |
| I3b | Tingling |
| I4a | Redness |
| I4b | Pain |

TABLE 9
Effectiveness Key

| | |
|---|---|
| E1 | No inflammation, no raised lesions |
| E2 | Reduced inflammation, reduced lesions |
| E3 | No Change |
| E4 | Increased lesions |

TABLE 10
A Results of Acne Treatment Round 1 and 2

| | 20-40 | | | |
|---|---|---|---|---|
| | Minutes | 1 Day | 3 Days | 1 Week |
| Subject T | | | | |
| A | I1ab 3, E3 | — | — | |
| B | I1ab3, E3 | I1, E3 | — | |
| B1 | I1ab, I2b, E3 | I1ab, E3 | I1ab, E 2 | I1ab |
| E 1-2 | | | | |
| Subject S | | | | |
| A | I1ab, I2b, E3 | I1ab, E2 | | |
| B | | | | |
| B1 | | | | |

### Example 2.3

### Pore Reduction and Cleansing

Background: Subject K is a 55 year old woman with large clogged pores on nose and forehead. She was treated with C1 twice a week. Treatment continues once a week to monthly to maintain attractive appearance.

Treatment (see table 5 below)

RESULTS: After 10 minutes, blackheads were lifted/scraped off with swiping motion of flat edged spatula designed for facial treatment. Pore size was reduced significantly. Impacted pores were clearer.

TABLE 11
RESULTS Pore Reduction and Cleansing

| | 20-40 | | 3 | |
|---|---|---|---|---|
| Subject K | Minutes | 1 Day | Days | 1 Week |
| C1 | I2A, B E1* | I2, E2 | I1, E1 | I1, E1 |

### Example 3

### Ingrown Toenail

Background: The subject is a 17-year-old male with a severely infected ingrown toenail. Hot water soaking of the foot did not improve the condition. Topical application of an antibiotic did not affect the condition of the infection. The consulting doctor recommended surgery.

TABLE 12
Compositions used to Treat Ingrown Toenail

| | | | 2.5% | | |
|---|---|---|---|---|---|
| | Benzoyl | Sodium | Glycolic | Miconazole | |
| | Peroxide | Chlorite | Acid | 2% | Antibiotic |
| A | 10% | 2% | | 1 part | 1 part |
| B | 10% | 1.8% | | | 1 part |
| C | 2.5% | 2r% | | 1 part | 1 part |

Treatment: Dec. 9, 2009: Treatment with A begins. Treatment is sporadic. The subject is a teenager. At least once a day and as often as twice a day, treatment A was applied. Use of a vinyl toe sock was attempted but subject complained of irritation. Treatment B is begun.

RESULT After 3 days: Periungal tissue began to separate from the ingrown side of the nail.

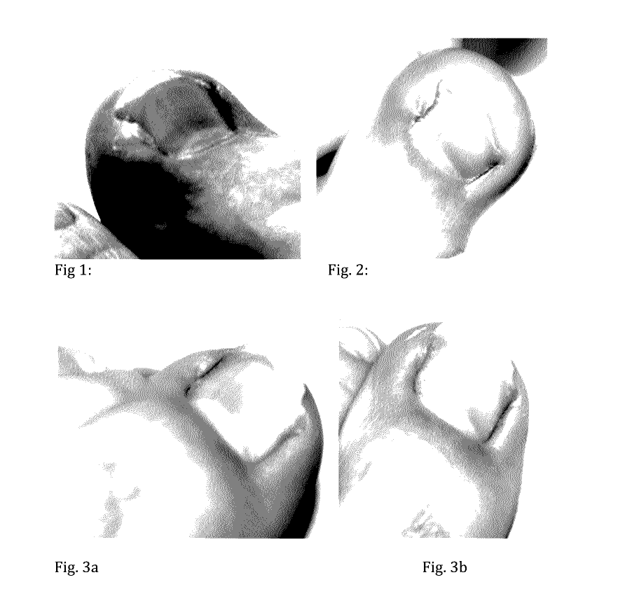

The foot was soaked in hot tap water 4 times; on Dec. 12, 2009, Dec. 15, 2009, Dec. 16, 2009, and Dec. 17, 2009. Infection began to drain. FIG. 1 illustrates the ingrown toenail prior to draining. FIG. 2 illustrates the ingrown toenail after two days of treatment, showing the skin separating from the nail. Recovery continued. After two weeks, treatment C is begun and continued intermittently (treatment once a day; 5 out of seven days). Good results continue though foot soaking is abandoned at start of treatment with C.

RESULT: Treatment was continued approximately twice a day for three days until skin separated and lifted at the severely infected inside corner of the ingrown toenail. At that point the infection began to drain when the foot was soaked in hot tap water on at least 4 separate occasions. FIGS. 3a and 3b illustrate the ingrown toenail after treatment.

### Example 4

### Keratosis

Subject is a 55-year-old woman. 3 groups of Keratosis were treated using various compositions.

TABLE 13
Keratosis Concentrations for Tested Treatments

| | | | 2.5% | Micon- | | |
|---|---|---|---|---|---|---|
| | Benzoyl | Sodium | Glycolic | azole | Anti- | Erithro- |
| | Peroxide | Chlorite | Acid | 2% | biotic | mycin |
| A | 10% | 2% | | | | 0.5 part |
| B | 2.5% | 1.5% | 1 part | | 1 part | 1 part |
| C | 10% | 2% U | 1 part | | 1 part | 1 part |
| C1 | 1.5% | 21.5% | 2 parts | | 2 parts | |
| D | 10% | 2.66% | — | | 1 part | 1 part |
| E | 10% | 1.5% | 1 part | — | — | |
| F | 2.5% | 2% r | — | — | — | |
| G | 2.5% | 2% r | 1 part | — | — | — |
| H | 2.5% | 2% r | — | — | — | —, 5 pt. |
| | | | | | | 5 mg/G |

No benzoyl peroxide, Glycolic acid, Miconazole
erythromycin, antibiotic was used in Example I.

| | Sodium | 1% | herbal |
|---|---|---|---|
| | Chlorite | Terbinafine | oil* |
| I. | 2% | 1 part | 2 parts |

*herbal oil consists of 3.736% each of thymol, menthol and menthyl acetate dissolved in 0.33% jojoba oil, 0.66% grapeseed oil and 7.654 g beeswax heated and added as thickener.

### Example 4

### Keratosis Examples

### Example 4.1

3 pinhead sized dots about ½ centimeter wide end to end. Entire grouping is raised and slightly reddened.

Treatment: Treated with a total of 3 applications of Treatment A.

RESULT: Brown spots remain. Raised skin is flattened.

### Example 4.2

One centimeter sprawling keratosis formation w/3 extremely distinct areas of separation.

Upon close inspection, a fourth separation can be perceived in the larger circular keratosis formation.

Treatment: Treated over a period of two months from Oct. 31, 2009 until Dec. 20, 2009 with abandonment of treatment Nov. 15, 2009 and its resumption on Dec. 14, 2009.

TABLE 14
Results of Various Compositions

| Treatments A, C | somewhat effective, though promising slow and |
|---|---|
| | somewhat irritating to skin |
| Treatment D | harsh and severely irritating and caused treatment |
| | to be dropped for one month |
| Treatment C | begun and was markedly effective for a three day |
| | course of treatment |
| Treatment B | repeated for two days with promising results |
| Treatment E | continues with Treatment E alone. Very quick |
| | effective results/somewhat dry skin |
| Treatment E | used with moisturizer. Very quick/gentle to the |
| | skin results |
| Treatment F | used. Low dose BP gentler to skin. Good result |
| Treatment G | continues. Good result, skin does not dry. Keratosis |
| | is reduced more effectively than w/F |
| Treatment H | quick perceivable reduction/flattening of Keratosis |
| | 4.2 |

### Example 4.3

On side of the nose, Keratosis darkened skin has characteristic barnacle-like look. Diagnosed Keratosis, Non-cancerous

TABLE 15
Results of Various Compositions

| | |
|---|---|
| Treatment E | Subject was treated a total of 3 times with Treatment |
| | E with good results |
| Treatment F | Low dose BP gentler to skin. Good result |
| Treatment F | resumed. Low dose BP gentler to skin. Good result |
| Treatment G | continues. Good result, skin does not dry. Keratosis |
| | color and high relief texture is reduced more |
| | effectively than w/F. |
| Treatment H | results in slight immediate redness/45 minutes |
| | later, slight reduction in color and texture of |
| | keratosis lesion |
| Treatment I | Division in growth followed by ⅔ falling off without scar |

### Example 4.4

On arm, 1 cm irregularly shaped previously diagnosed keratosis growth began changing color from dark brown to lighter tones. Treatment I was applied to ½ of the growth twice, morning and evening. ⅔ of the Keratosis growth fell off within 2 days leaving clear unscarred skin beneath. Biopsy of remaining ⅓ growth tested benign for cancer.

### Example 5

A mixture of 2.07% sodium chlorite, tea tree oil and an azole (2% Miconazole) were rubbed onto the left heel for twenty days. The right heel remained untouched and served as a control. After twenty days, skin was evaluated for softness, coarse roughened skin, and white deposits in the cracks of skin. The heel treated with composition was markedly softer and appeared healthier, with smoother appearance.

### Example 6

### Anti-Candidiasis Treatment

An in vitro determination of the use of sodium chlorite on human tissue culture and broth culture of Candidia albicans was performed. Concentrations of 2% and 0.5% were both effective in killing vegetative Candidia albicans liquid culture. Continued culture without sodium chlorite did not result in regrowth of Candidia albicans suggesting cell killing and (if present) spore inactivation rather than simple growth inhibition.

When tested against human cells in tissue culture, 2% sodium chlorite was shown to be marginally toxic while 0.5% did not display toxicity to the human cells.

TABLE 16a
Effects of Sodium Chlorite on Candidia albicans

| Concentration of | | |
|---|---|---|
| Sodium Chlorite in | | Candidia albicans |
| Culture Broth | Toxicity | killing |
| 0.5% | None | Yes |
| | observed | |
| 2.0% | Toxic | Yes |

Additional testing was performed using dilutions of sodium chlorite and discoloured alone and in combination. The data was given in FIG. 19. Growth of Candidia albicans was determined by optical adsorption.

### Example 7

Various herbal compositions and sodium chlorite were applied to skin and/or nails.

Methods and Materials:

In one method of making the products of the invention, menthol was added to grapeseed oil and set aside capped in a cool dark place for approximately 24 hours. At that point, menthol crystals had liquefied in the oil base. Next, thymol was heated in the menthol/grapeseed oil composition to 52 degrees C., at which point it liquefied. 0.25 oz of pharmaceutical grade beeswax was added to the composition and heated to 66 degrees C. Resulting composition was poured into sterile containers. Upon cooling, mixture resembled the consistency of petroleum jelly. A pleasant odor emits from the composition.

In a further method of making the products of the invention, 3.800 grams each of thymol, menthol and menthyl acetate were added to 0.66 grapeseed oil and 0.33 jojoba oil and set aside capped in a cool dark place for approximately 24 hours. At that point, thymol and menthol crystals had liquefied. 0.25 oz of pharmaceutical grade white beeswax was added to the composition and heated to 66 degrees C. Resulting composition was poured into sterile containers. Upon cooling, mixture resembled the consistency of petroleum jelly. A pleasant odor is emitted from the composition.

Preparations:

E. A solution containing 2.0% SC in water solution was dropped onto each infected toe. This applied with 1 drop of 100% olive oil on the toenail and rubbed into toenail briefly.

F. A solution containing 2% sodium chlorite and water was combined with 100% olive oil in a ratio of 1.5 parts of the 2% sodium chlorite solution to 1 part olive oil. Upon shaking, bubbles appeared then resolved into what appeared to an emulsion. The resulting substance was applied to each infected toe.

G. 1.5% thymol alone in glycerin (1.5% T+G) A solution containing 1.5% heat liquefied thymol (crystals) in glycerine was prepared. The composition was applied to each infected toe.

G*. 1.5% SC was applied to affected nail followed by application of Composition G.

H. 2% thymol alone in glycerin (2% T+G) composition containing 2% heat liquefied thymol crystals in glycerine was prepared. The resulting substance was applied to each infected toe.

H*. 2% sodium chlorite was applied to affected nail, followed by application of Composition

I. (2% T+GS) A solution containing heat liquefied thymol from crystals in were combined with grape seed oil to produce composition containing 2% thymol. The resulting substance was applied to each infected toe after application of 2% sodium chlorite by dropper to each affected toe.

J (T+M+GS) A solution containing 2% Menthol crystals liquefied in grape seed oil was combined with 2% heat-liquefied Thymol crystals in grape seed oil. The resulting substance was applied to each infected toe after application of 2% sodium chlorite by dropper.

K. (T+GS+Beeswax) A solution containing 2% heat liquefied thymol crystals was combined with grape seed oil then melted in beeswax (3 oz beeswax in 100 ml of thymol/Grapeseed oil). The resulting substance was applied to each infected toe after application of 2% sodium chlorite by dropper to each affected toe.

L. (M+GS+Beeswax) A solution containing 2% menthol crystals liquefied in grape seed oil, then melted with beeswax (3 oz beeswax in 100 ml of menthol in grapeseed oil). The resulting substance was applied to each infected toe after application of 2% sodium chlorite by dropper.

M. (T+M+GS+Beeswax) A solution containing 2% menthol crystals liquefied in grape seed oil was combined with 2% heat liquefied thymol crystals in grape seed oil, then mixed with melted in beeswax (3 oz beeswax in 100 ml of Grapeseed oil). The resulting substance was applied to each infected toe.

M1. (T+M+GS+Beeswax) A solution containing 2% menthol crystals liquefied in grape seed oil was combined with 3% heat liquefied thymol crystals in grape seed oil, and then mixed with melted in beeswax (3 oz beeswax in 100 ml of Grapeseed oil). The resulting substance was applied to each infected toe after application of 2% sodium chlorite by dropper.

M2. (T+M+GS+Beeswax) A solution containing 2% menthol crystals liquefied in grape seed oil was combined with 3% heat liquefied thymol crystals in grape seed oil, then mixed with melted in beeswax. 0.01% Tocopheryl acetate was added. The resulting substance was applied to each infected toe after application of 2% sodium chlorite solution.

M*. 2% SC was applied to each infected toe followed by Composition M.

M4. (T+M+GS+Beeswax) A solution containing 4% menthol crystals liquefied in grape seed oil was combined with 2% heat liquefied thymol crystals in grape seed oil, then mixed with melted in beeswax (3 oz beeswax in 100 ml of Grapeseed oil). The resulting substance was applied to each infected toe after application by dropper of 2% sodium chlorite solution.

N. (T+OO) A solution containing 2% heat liquefied T\thymol crystals were combined with olive seed oil. The resulting composition was applied to each infected toe after application of 2% SC by dropper to each affected toe.

O. (T+M+OO) A solution containing 2% menthol crystals liquefied in olive oil was combined with 2% heat-liquefied thymol crystals in 100% olive oil. The resulting substance was applied to each infected toe after application of 2% SC to each affected toe.

P. 1.5% thymol and 1.5% Menthol liquefied in 100% canola oil are applied to each affected toe after 1.5% sodium chlorite is applied.

Q. Up to 100% thymol crystals are combined with an extender that does not adulterate the chemical properties of the thymol (such as but not limited to corn starch) to make a mixture. The resultant mixture is lightly packed under the affected nail.

Q*. 1-2% sodium chlorite is applied before or after application of Q.

R. Thymol crystals are combined with an extender that does not adulterate the chemical properties of the Thymol (such as but not limited to corn starch). The resultant mixture is lightly packed under each affected nail. Next, Composition M is smoothed over the entire nail, periungal tissue and cuticle. In the process, M is glided over the dry thymol mixture to act as a seal. Compositions K and L can be substituted for M. Method R can be alternated with Compositions K, L, and M*; for example: Method R in the morning and Method M* in the evening

S. 2% sodium chlorite was applied to affected nail. Next 5% vinegar solution in water was applied by dropper. Composition M was applied next.

S1. 2% sodium chlorite was applied to affected nail followed by 5% acetic acid solution.

T. 2% sodium chlorite was applied by dropper followed by 2% Thymol heat liquefied in Mineral oil applied by dropper to fingernail.

U. 2% sodium chlorite was applied by dropper followed by 100% raw unrefined “active+15” Manuka honey was applied to nail.

V. 2% sodium chlorite was applied by dropper followed by Manuka honey which was mixed with Composition M before use and stored in container. Ratio was 1:1. Manuka honey: Composition M.

W. 2% sodium chlorite was applied by dropper followed by a composition as described: Ingredients according to M4 were prepared and cooled. During “cool down” the mixture was continually stirred. When temperature had dropped to around 97 degrees, Manuka honey was added. Ratio 1:1; M4 composition: 1 Manuka honey: 1. Also during cool down a solution containing 0.04 g of (5,746 IU) d alpha tocopheryl acetate was blended into the mixture.

X. Composition M was prepared. During cool down a solution containing 0.04 g of (5,746 IU) d alpha tocopheryl acetate was blended into the mixture, stored then applied to nail after application of 2% sodium chlorite.

Y. Composition M was prepared. During cool down a solution containing 2% Rosemary oleoresin was blended into the composition. Ratio 1 ml Rosemary Oleoresin: 100 ml Composition M.

Z. 2% sodium chlorite solution was first applied by dropper followed by a solution containing 2% heat liquefied Thymol and 5% acetic acid solution in water and applied to nail.

Za. 2% sodium chlorite and coconut oil

Zb. 2% sodium chlorite by dropper followed by coconut oil and 3% heat liquefied menthol.

Zc. 2% sodium chlorite by dropper followed by coconut oil and 3% heat liquefied thymol and 2% menthol in beeswax.

Zd. Composition Zc with introduction of vinegar which contains 5% acetic acid between the use of sodium chlorite by dropper and the coconut oil beeswax/carnauba blend.

1. (T+M+MA+GS+Beeswax) A solution containing 2% menthol crystals and 2% menthyl acetate and 100 ml of grape seed oil was left overnight, then combined with 2% heat liquefied thymol crystals, then mixed with melted 0.57 oz. beeswax. 0.01% Tocopheryl acetate was added. The resulting substance was applied to each infected toe after application of 2% sodium chlorite solution.

2. (M+MA+GS+Beeswax) A solution containing 2% menthol crystals liquefied in 2% menthyl acetate and 100 ml of grape seed oil was left overnight, then mixed with melted 0.57 oz. beeswax. 0.01% Tocopheryl acetate was added. The resulting substance was applied to each infected toe after application of 2% sodium chlorite solution.

3. (T+M+MA+GS+Beeswax) A solution containing 2% menthol crystals liquefied in 4% menthyl acetate and 100 ml of grape seed oil was left overnight then combined with 2% heat liquefied thymol crystals, then mixed with melted 0.56 oz. beeswax. 0.01% Tocopheryl acetate was added. The resulting substance was applied to each infected toe after application of 2% sodium chlorite solution.

4. (T+M+MA+GS+Beeswax) A solution containing 3% menthol crystals liquefied in 3% menthyl acetate and 100 ml of grape seed oil was left overnight then combined with 2% heat liquefied thymol crystals, then mixed with melted 0.6 oz. beeswax. 0.01% Tocopheryl acetate was added. The resulting substance was applied to each infected toe after application of 2% sodium chlorite solution.

5. (T+M+MA+GS+Beeswax) A solution containing 2% menthol crystals liquefied in 4% menthyl acetate and 100 ml of grape seed oil was left overnight then combined with 2% heat liquefied thymol crystals, then mixed with melted 0.57 oz. beeswax. 0.01% Tocopheryl acetate was added. The resulting substance was applied to each infected toe after application of 2% sodium chlorite solution.

6. (T+M+MA+GS+Beeswax) A solution containing 2.70 g menthol crystals liquefied in 1.80 g menthyl acetate and 100 ml of grape seed oil was left overnight then combined with 2.70 g heat liquefied thymol crystals, then mixed with melted 0.57 oz. beeswax. 0.01% Tocopheryl acetate was added. The resulting substance was applied to each infected toe after application of 2% sodium chlorite solution.

7. (T+M+MA+GS+Beeswax) A solution containing 2.70 g menthol crystals liquefied in 1.80 g menthyl acetate and 100 ml of grape seed oil was left overnight then combined with 2.70 g heat liquefied thymol crystals, then mixed with melted 0.57 oz. beeswax. 0.01% Tocopheryl acetate was added. The resulting substance was applied to each infected toe after application of 2% sodium chlorite solution.

8. (T+M+MA+GS+Jojoba oil+Beeswax) A solution containing 3.736 g each thymol and menthol crystals were liquefied in overnight in a closed container with 0.33 g jojoba oil, 0.66 grapeseed oil, and 3.736 g menthyl acetate then combined with melted 7.654 g. melted beeswax. The resulting substance was applied to each infected toe after application of 2% sodium chlorite solution.

9. Composition 8 was used with 1% terbinafine

10. Composition 8 was used with 1% clotrimazole herbal carrier base containing BHT, Safflower oil, Tinctorius, Emu oil, Eucalyptus Globulus oil, Soybean oil, Lavender oil, Teatree oil, peppermint oil, olive oil, sweet Almond oil, Rose Hips oil, Jojoba oil, Tocopheryl, Acetate, urea

11. Composition 8 was used with 10% efinaconazole.

12 Composition 8 was used simultaneously with Tavaborole, (Kerydin™)

13. 8. (T+M+MA+GS+Jojoba oil+Beeswax) A solution containing 3.80 g each thymol and menthol crystals were liquefied in overnight in a closed container with 0.33 g jojoba oil, 0.66 grapeseed oil, and 3.80 g menthyl acetate then combined with melted 7.654 g. melted beeswax. The resulting substance was applied to each infected toe after application of 2% sodium chlorite solution.

Carnauba wax, shea butter or petroleates can be added to beewax in compositions or substituted for beeswax in compositions that contain beeswax. Cosmetic grade Colorants may be added to mixtures.

Compositions K or L or M or M1 can be used interchangeably with compositions involving M or M1.

One may first apply a composition to at least one nail infected with nail fungus multiple times over a period of at least three days followed by applying a second composition for treating or preventing nail fungus, followed by a third and fourth wherein said first composition and said second composition-fourth composition differ in their chemical components. Example: First Composition K is used 7 days, next L, then M1, then S.

Petroleum jelly may be substituted for Beeswax. Beeswax is exemplified. Any Oil can be substituted for Grapeseed oil in Compositions K, L, M, M1. Most preferred are oils that readily release peroxides when oxidized in the presence of the chlorite. Olive oil and Sunflower oil are non-limiting examples. Subjects were instructed to treat themselves twice a day. Compliance varied from stringent to moderate. One subject only applied compositions once a day and still steadily improved

Except for Composition Q, which was tested on a lightly infected fingernail, all compositions were tested on thickened discolored toenails indicating presence of a bacterial or fungal infection. All subjects had been diagnosed with onychomycosis by a podiatrist or dermatologist prior to beginning the study. Infections ranged from light (only distal portion of nail affected) to severe (entire nail affected). One subject of 7 had lightly infected nails. With all others at least 70% of the nail was infected.

Tables 16 provides the ratings of fungus quality. Table 17 provides the ratings of skin roughness, which reflects the compound's effect on fungus impregnated skin around the nails and cuticles. Table 18 provides the ratings for healthy nail appearance. Table 19 provides the ratings for irritation of nail and skin. Table 20 provides an evaluation of the compositions in terms of fungus quality, skin roughness, healthy nail appearance, and irritation of nail and skin.

TABLE 16
Fungus Quality

| | |
|---|---|
| 1 | Fungus is reduced, is easily removed, |
| | nail flattens |
| 2 | Improvement |
| 3 | No Change |
| 4 | Relapse. Formerly flakey, easy- to- |
| | remove fungus transforms back into |
| | a unified, hard, difficult to penetrate |
| | plastic-like wall |

TABLE 17
Skin Roughness

| | |
|---|---|
| 1 | Improvement |
| 2 | No Change |
| 3 | Slight Skin Roughness Observed |
| 4 | Severe Skin Roughness is Observed |

TABLE 18
Healthy nail appearance

| | |
|---|---|
| 1 | Dramatic improvement |
| 2 | Improvement |
| 3 | No change |
| 4 | Relapse. The white edge of the nail |
| | widens. Lateral striations |
| | originating at the nail edge begin to |
| | intrude on the body of the nail. |

TABLE 19
Irritation of Skin and Nail

| | |
|---|---|
| 1 | Feel nothing |
| 2 | Slight pleasant tingle |
| 3 | Discomfort |
| 4 | Whenever Stage 4 occurs, or an |
| | irritation level 3 is reached, testing of |
| | listed Composition was abandoned |

TABLE 20
Evaluation of Compositions

| | Fungus | Skin | Nail | |
|---|---|---|---|---|
| Treatment | Quality | Roughness | Appearance | Irritation |
| E | 1 | 1 | 1-2 | 1 |
| F | 1 | 1 | 1-2 | 1 |
| G | 3-4 | 2 | 4 | 1 (S) |
| G* | 1 | 1 | 1 | 1(L) |
| H | 3 | 1-2 | 3 | 1(S,k,L,J2) |
| H* | 2 | 1 | 2-3 | 1 (L:3J2:3S2) |
| I | 2 | 1 | 2 | 1(k) |
| J | 1 | 1 | 1.5 | 1(LJ2) |
| K | 1-2 | 1 | 1-2 | 1 J3,K |
| L | 2 | 1 | 2 | 1 (J3) |
| M | 1-2 | 1 | 2 | 1 (J3) |
| M* | 1 | 1 | 1 (J3, k) | 1 |
| M1 | 1 | 1 | 1 (J3, k, T) | 1 |
| M2 | 1 | 1 | 1 (J3, k, T) | 1 |
| N | 1 | 1 | 1-2 | 1(J2, L) |
| O | 1 | 2-3 | 1-2 | 1(J2, L) |
| P | 1 | 1 | 1 | 1 (S) |
| Q | 2-1 | 2 | 1 | 2 |
| R | 1-2 | 2 | 1 | 2 |
| S | 1 | 1 | 1 | 2 |
| S1 | 2-1 | 2 | 2 | 2 |
| T | 2-3 | 2-1 | 2-3 | 2 |
| U. | 1-2 | 1 | 1 | 1-2 |
| V. | 1-2 | 1 | 1 | 1-2 |
| W | 2 | 1 | 2-3 | 2 |
| X | 2-1 | 1-2 | 2 | 2 |
| Y. | 2-1 | 2-1 | 4(discoloration) | 2 |
| Z. | 2-3 | 2-1 | 3 | 1 |
| Za | 2 | 1 | 2 | 1 |
| Zb. | 2-1 | 1 | 2-1 | 1 |
| Zc. | 1-2 | 1 | 1-2 | 1 |
| Zd. | 1-2 | 1 | 1 | 2 |
| 1. | 2 | 1 | 1-2 | 1-2 |
| 2. | 1 | 1 | 1 | 1-2 |
| 3 | 3 | 1 | 3 | 1-2 |
| 4. | 3 | 1 | 3 | 1-2 |
| 5. | 3 | 1 | 3 | 1-2 |
| 6. | 3 | 1 | 3 | 1-2 |
| 7. | 2+ | 2 | 2+ | 1-2 |
| 8. | 1 | 1 | 1 | 1-2 |
| 9. | 1 | 1 | 1 | 1-2 |
| 10. | 1 | 1 | 1 | 1-2 |
| 11J | 2+ | 2+ | 2+ | 1-2 |
| 12 | 1 | 1 | 1 | 1-2 |
| 13. | 1 | 1 | 1 | 1-2 |

Observations:

A combination of thymol in glycerin and thymol alone were the poorest performers against nail fungus as compared to thymol in glycerine immediately after application of sodium chlorite.

A lightly infected nail was treated with thymol in glycerin after a drop of 2% sodium chlorite. Nail was cleared within 17 days. At that point, sodium chlorite was removed from the treatment regimin and thymol in glycerine alone was used for 7 days. The nail began to darken indicating relapse. Sodium chlorite solution was again included in the treatment plan. After 14 days, the nail appears clear again. Hence, it appears sodium chlorite solution application improves the response to the thymol preparations against nail fungus. When sodium chlorite solution was used in combination with thymol and/or menthol in an oil base, with and without beeswax, notable Effects were perceived within 1 week. The addition of Beeswax or a petrolatum improved all compositions with which it was tested, with composition M1 exemplified.

A comparison was performed of the comparative effectiveness of several compositions containing the same quantities of sodium chlorite, beeswax, and grapeseed oil. The distinction in the compositions was that:

Composition M contains 2% thymol and 2% menthol.

Composition K contains 2% thymol.

Composition L contains 2% menthol.

The superior result was achieved with composition M, followed by K and L in that order. composition M which contains 2% thymol and 2% menthol in an oxidizable oil (in this case grapeseed oil) combined with beeswax was surprisingly effective without sodium SC, but it was less effective than Composition M1 and K, and slightly less effective than Composition L which also contained SC.

In one instance, the subject first applied 2% sodium chlorite to each affected nail by dropper, taking care to cover entire nail surface, cuticle, periungal tissue and under the nail. Within minutes to immediately, the composition containing Thymol, Menthol, Grapeseed oil and Beeswax was applied to surface of nail, cuticle, periungal tissue and under the nail. Subject repeated application morning and evening. Result: Nail lightened, discoloration lifted. Hard fungus softened. Nail became less brittle. Though not completely cleared, moderately-severely infected nails were notably aesthetically improved within one week.

Compositions used were applied to two 4 toenails that had long cuticles covering at least one third of the nail. After application, the dead over-long cuticle separated from healthy cuticle, leaving an aesthetically pleasing thin edge surrounding the nail. No tearing of healthy cuticle occurred. No mechanical implements such as cuticle scissors were used to achieve these beneficial effects.

Symptoms of itching and rash characteristic of Tinea Pedis between toes and on soles of feet ceased after three applications of composition I. An early outbreak of Tinea Pedis symptoms ceased after two treatments with M1 and K.

Notable cosmetic improvement of the appearance of the nail cuticle and surrounding skin with compositions as noted in data chart under skin roughness. No cuticle scissors were used and dead rough skin resolved within one week with oil or oil and beeswax-based compositions. As treatment with the sodium chlorite and thymol and/or menthol compositions began and progressed, the reduction of the most prominent corn (which was surmounted by a white hardened pimple atop raised reddened skin) continued to progress rapidly over the next two weeks. In two corns, skin is only slightly reddened. Callusing is gone. Reduction of corns is significant. A third corn was surmounted by a “pimple.” Treatment was applied, then abandoned. Patient had corn mechanically reduced by his podiatrist who told him the corn would come back. When the pimple began to form again, treatment was applied again. Corn reduced in size, pimple disappeared.

Twice daily treatment with Composition M1 was effective at softening a plantar wart. Black center of wart is gone or lightened and heavy callused skin encircling the formerly black center is soft and appears to be flatter.

Dry skin and calluses are softened by application of Compositions to whole foot.

A lesion on a foot was characterized by deep fissures in the skin before treatment. Treatment with a sodium chlorite and clotrimazole composition followed by olive oil in a 1:2 ratio was applied once per day for two days. One additional treatment was applied and then two treatments were applied every other day with 2% sodium chlorite alone followed by twice as much of a mixture of olive oil and grape seed oil.

Acceleration of nail growth has been reported by 4 subjects regarding nail treatments that contain sodium chlorite in a several step system or one step system of application.

In two instances, a subject's irritation from insect bites was relieved by application of sodium chlorite and activated carbon. Approximately 12 hours after being bitten by a small flying insect (species undetermined) a bulls-eye inflammation pattern resulted that was 2.25 inches in diameter. 2 drops of 2% Sodium chlorite solution were dropped onto activated carbon powder to form a paste. A quarter sized amount was applied to central bite area. Itching ceased immediately. A band-aid was next applied. Itching immediately ceased. 90 minutes later, wound site was cleansed. Red inflamed “bull's eye pattern was entirely gone leaving a slightly raised bump about 6 mm in diameter. No itching was reported after treatment with the composition described herein.

The next morning treatment was repeated. Poultice was removed after 45 minutes.

A spider bite was also treated with a combination of sodium chlorite and activated carbon in a beeswax mixture containing 3% thymol. Immediate cessation of itching is reported along with marked reduction in inflammation.

### Example 8

### First Aid Wound Treatment

5-5-12: Knife injury bled copiously.

5-6-12: Wound was initially washed and treated with peroxide and a very tight bandage. 17 hours after the injury, the wound still hurt and was still “opening up” and bleeding from time to time evidenced by fresh blood 17 hours later. At 17 hours, injury was treated w/Thymol in beeswax preparation with sodium chlorite. No menthol was used. There was no discomfort at initial application of composition.

5-7-12: 24 hours after thymol treatment. Treatment reapplied, slight sting was felt upon application—nothing excessively painful.

5-8-12: Treatment was reapplied after application of peroxide.

5-9-12: 3 days after initial treatment (72 hours) 4 days after initial injury, wound edges are healed evenly with skin and are reduced 85-90%.

Other compositions which combine a solution, cream, gel, or lotion consisting of around 0.005%-5%, preferably 3% peroxide, preferably but not limited to, hydrogen peroxide and a sodium chlorite and water solution, cream gel or lotion at a ratio of 1 part peroxide to 2 parts sodium chlorite solution may have similar affects. At 1:1-1:8 ratios of peroxide to sodium chlorite solution benefit may be obtained. This composition can be used alone or with antibiotics or other beneficial agents to increase their penetration to aid treatment of bacterial and fungal infections of the skin and nails. A pH of around 3-12, preferably around 10, is preferred.

Another preferred method of the aforementioned embodiments combines a solution, cream, gel, or lotion consisting of any concentration of olive oil with compositions containing chlorite salts. While there are other chlorite salts that may be used, sodium chlorite as exemplified is preferred and economical. Preferred compositions contain sodium chlorite in any concentration, but most preferred are those at at between 0.75-3:0%, most preferably around 2% of the active agent.

Another method of the aforementioned embodiments combines a solution containing one or more of the following components in a cream, gel, or lotion or liquid: such as but not limited to mineral oil, glycerin, petroleum jelly, petrolatum, beeswax, shea butter, carnauba wax, propylene glycol, diethyl phthalate, Ethoxydiglycol, Hydroxypropylcellulose, alcohol, acetic acid, and thymol and/or menthol and/or camphor used in combination with sodium chlorite or compositions containing chlorite salts.

While there are other chlorite salts that may be used, sodium chlorite as exemplified is preferred and economical. Preferred compositions contain sodium chlorite in any concentration, but must preferred are those at at between 0.75-3:0%, most preferably around 2% of the active agent. Carrier oils such as but not limited to grapeseed and olive oil, and natural bases such as but not limited to beeswax and shea butter may be combined with the forementioned components.

Antioxidants such as but not limited to d-alpha tocopheryl acetate may be used in this invention. The non-limiting preferred ratio is Vitamin E 0.01: Compositions A-X: 100.

The products of the invention may be packaged containing sodium chlorite solution and other components packaged separately in kit form for mixing and or simultaneous application at the time of use.

This invention may offer a method for delivering the benefits of ozonated oils quickly and cheaply with the addition of sodium chlorite immediately before use. The advantage over the use of the pre-mixed materials of the prior art is that, unlike the ozonated oils which need refrigerating, the components in the kit need not be refrigerated.

A liquid composition, cream, gel, or lotion containing or combined with for example, mineral oil, glycerin, petroleum jelly, petrolatum, beeswax, shea butter, carnauba wax, propylene glycol, diethyl phthalate, Ethoxydiglycol, Hydroxypropylcellulose, alcohol, acetic acid with any concentration of sodium chlorite, preferably about 1% to 10%, most preferably around 2% sodium chlorite, can also be provided for use with antibiotic or other active agents with sodium chlorite providing enhanced penetration of the antibiotic or other active agents where product is left on from 3 minutes to several hours. To the compositions of the invention there may also be added other antimicrobial agents such as antibiotics. The use of beeswax has proven to be particularly beneficial.

Alpha hydroxy acids are well known for use in the skin treatment and may be used with compositions of the present invention. Representative alpha hydroxy acids include mandelic acid, lactic acid, glycolic acid, etc. and salts thereof. One or more of these (or their salts) may also be used to serve as a pH adjusting agent or a buffering agent. While citric acid/citrate buffer is typically used, other pH-adjusting agents or buffering agents may be used alone or in combination with the alpha hydroxy acids of the present invention. Concentrations in the range of about 0.1% to about 20% are preferred. Depending upon the specific compound used and/or the intended frequency of use, narrower ranges are preferred. More preferred are ranges such as 0.2%-7% glycolic acid for daily use. Alpha hydroxyl acids, particularly glycolic acids are particularly effective for cuticle treatments when combined with above 2% sodium chlorite.

Further, the present invention may include a polyphosphate as a whitening aid agent and as a stabilizer for sodium chlorite and/or peroxide. Examples of a polyphosphate which can be used according to the present invention includes one or more selected from a group consisting of tetrasodium pyrophosphate (TSPP)(preferred), sodium acid pyrophosphate (SAPP), sodium hexametaphosphate (SHMP), sodiumtripolyphosphate (STP), sodium potassium tripolyphosphate (SKTP), tetrapotassium pyrophosphate (TKPP), ultraphosphates such as acidic sodium metapolyphosphate and acidic sodium polyphosphate. Polyvinyl pyrrolidone, Polyquaternium-11, polyquaternium-39, polyvinyl pyrolidone-vinyl acetate copolymer (PVP/VA copolymer) can be used according to this invention.

Other suitable polymers may be one or two or more kinds selected from hydroxypropylmethyl cellulose, hydroxyethyl cellulose, polyvinyl alcohol, hydropropyl cellulose, carbomer, a copolymer of methyl vinyl ether and maleic anhydride and polyethylene oxide.

Calcium peroxide, carbamide peroxide, sodium percarbonate, sodium perborate, tetrasodium pyrophosphate peroxidate and mixtures thereof can be substituted for hydrogen peroxide.

The invention may contain one or more cosmetically approved solvents which may include monoalcohols such as alkanols having 1-8 carbon atoms (ethanol, isopropanol, benzyl alcohol, phenylethyl alcohol, polyalcohols such as alkylene glycols (glycerine, ethylene glycol, and propylene glycol) and glycol ethers such as mono-di and tri-ethylene glycol monoalkyl ethers; for example ethylene glycol monomethyl ether and diethyleneglycol monomethyl ether used alone singly or in a mixture 0.1-72% by weight relative to the weight of the whole composition.

Other optional ingredients include the use of diluents, salts to make the compositions isotonic, solvents, emollients, humectants, pH buffers, beta hydroxy acids, thickeners, binders, stabilizers, emulsifiers, abrasives, anti-inflammatory agents and chemicals involved in generating chlorine dioxide such as a metal salt of hypochlorite.

The final form of one or more components to the composition of the present invention is preferably liquid or spreadable solid (paste, gels, powders, ointments, etc.). A soap product containing some of the components of the composition of the present invention may be formed. This soap may be either in liquid or solid bar form. Components that are incompatible or volatile may be provided separately such as in a dropper bottle. In such a way a person may wash and simultaneously treat infected nails or prophylacticly apply some or all of the components to the nails, and if necessary followed by application of one or fewer liquids to the nails. Optionally, one may formulate a treatment bar with a different solidifying agent that resembles a soap bar but without the active soap ingredient. One example would be with the use of stearyl alcohol as the solidifying agent.

In an embodiment, chlorite salt compounds may be applied directly to the infected nails, nail bed and surrounding skin. The sodium chlorite compound may be applied to the nail in any form, but it is preferred that the compound is in solution or emulsion form. Any chlorite salt or chlorine dioxide compound that negatively affects the presence of onychomycosis in the finger and toenails may be used in the present invention.

Compositions used in the present invention include those in one container and those prepared from multiple containers. The nature of some of the chemicals used makes them react with each other over time resulting in a product, which lacks one-year (or greater) shelf stability. In such a situation, the present invention may be a kit containing two or more containers of chemicals such that chemicals reactive with each other are kept in separate containers. Compounds that are stable together are preferably premixed. The contents of the containers may be mixed immediately prior to use, added simultaneously or sequentially at the target site of infection or may be mixed together ahead of time and briefly stored before use. For example, some components may be mixed in liquid form and stored for example, about two months or so whereas the original components may be shelf stable for over one year. Storage under refrigeration or freezing conditions may extend the acceptable storage time. The final kit may include packaging for the two or more containers and instructions for preparation and use. Individual containers may contain any form of solids, liquids, gels, ointments, creams, etc. At least one of the containers or an additional empty container may be resealable after dispensing its contents after each application.

More specifically, one may use a two (or more) barrel syringe or other multichambered container to co-extrude the different composition components which are either mixed immediately before applying to the nail or are applied simultaneously or sequentially on the skin or nail.

An alternative container may be a multipack containing individual dosages of one or more of the containers. For example, a bottle containing capsules (or other individual dose container) where each capsule contains one or more components of the composition of the present invention; for example, capsules that may be punctured for administration to the target area. Different bottles may contain different capsules and different capsules may be opened immediately before use.

The components may also be held in the form of an emulsion with the active ingredients in one or more discontinuous phases in the emulsion. Water-in-oil emulsions are particularly preferred. This allows reactive components to be kept apart until mixed and applied. Furthermore, judicious selection of the continuous phase may entrap volatile or reactive compounds away from each other leading to a longer shelf life.

While olive oil and grapeseed oil has been most extensively exemplified, other component such as sunflower oil, safflower oil, coconut oil, or any other oil known to a practitioner of the art as “carrier oils” may be practiced in this invention and/or Manuka honey and/or any “active” honey which contains glucose oxidase. It is further considered that any oil or product that is oxidizing may be used with the chlorite in the method of the invention. In some instances, it might be beneficial to use oil that has a disagreeable odor. For example, a product that gives an odor that is offensive to a pet may keep the animal from licking the treating mixture.

Note: Treatments described herein promote healthy disease-free nail growth but do not rely on nail growth as a measure of positive results. Since beneficial compositions are penetrating the nail to the nail bed, results are immediately apparent. The nail acts as a translucent window through which one can observe the presence of fungus and the reduction of fungus. Even portions of the nail that are discolored and distorted by the presence of fungus return to an aesthetically pleasing normal healthy color, form and texture after consistent use of the treatments described herein.

### Example 9

Composition 1. (T+MA+glycerin) A solution containing 3.66 g thymol crystals was liquefied in 3.66 g menthyl acetate and combined with 100 ml of glycerin. The resulting substance was applied to each infected toenail after application of 2% sodium chlorite solution.

Composition 2. (T+MA+GS+Beeswax) A solution containing 3.66 g thymol crystals and 3.66 g menthol crystals were liquefied in 3.66 g menthyl acetate in 66 ml of grape seed oil and 33 ml of Jojoba oil, then combined with 0.30 oz. melted beeswax. The resulting substance was applied to each infected toenail after application of 2% sodium chlorite solution.

Composition 3. (M+MA+GS+Beeswax) A solution containing 3% menthol crystals dissolved in 3% menthyl acetate and grape seed oil and then mixed with melted 0.57 oz. beeswax. 01% Tocopheryl acetate was added. The resulting substance was applied to each infected toenail after application of 2% sodium chlorite solution.

Composition 4. (M+MA+glycerin) A solution containing 3% Thymol was dissolved in glycerin. The resulting substance was applied to each infected toenail after application of 2% sodium chlorite solution.

Composition 5. 2% sodium chlorite and 1% Tolnaftate composition. Substances were mixed then applied.

Composition 6. Composition 16 was mixed in hand with 10% efinaconazole then applied to areas affected by onychomycosis (Table 14), dyshidrotic excema (see Table 1) and psoriasis (see Table 2). Appearance of skin and nails improved.

Composition 6a. 2% sodium chlorite solution at pH6 was combined with efinaconazole on the nail. Appearance of nails improved within one week.

Composition 7. 2% Sodium chlorite and 2% Clotrimazole were combined, then applied to areas effected by onychomycosis, dyshidrotic excema (See Tablel) and psoriasis (see Table 2). Symptoms of scaling, rash and discolorations were significantly reduced. Appearance of skin and nails improved.

Composition 8. Composition 2 was mixed in hand with 2% Clotrimazole then applied to areas effected by onychomycosis and psoriasis (See Table 2). Appearance of skin and nails improved.

Composition 9. Composition 2 was mixed in hand with 5% Tavaborole then applied to areas effected by onychomycosis. Appearance of skin and nails improved.

Composition 9a. 2% sodium chlorite with Glycolic acid to adjust pH to 6 (which does not generate effective amounts of chlorine dioxide) was applied immediately after 5% Tavaborole. Appearance of nails improved within ten days.

Composition 10. Sodium chlorite was mixed in hand with 10% Efinaconazole and then applied to areas effected by psoriasis (see Table 2), dyshidrotic excema (see Table 1) and onychomycosis.

Composition 11. 2% Sodium chlorite and 2% Clotrimazole were combined, then applied to areas effected by onychomycosis and psoriasis (see Table 2). Symptoms of scaling, rash and discolorations were significantly reduced. Appearance of skin and/or nails improved.

Composition 12. 2% Sodium chlorite and 2% Clotrimazole were combined, then applied to areas effected by onychomycosis. Symptoms of nail scaling and discolorations were significantly reduced. Appearance of skin and nails improved.

Composition 13. 2% sodium chlorite and water solution was combined with 100% olive oil in a 1; 1 ratio and then applied to nails diagnosed with onychomycosis. Appearance of nail improved.

Composition 14. (T+MA+GS+Beeswax) A solution containing 3.736 g thymol crystals and 3.736 g menthol crystals and 3.736 g of menthyl acetate were liquefied in 66 ml of grape seed oil and 33 ml of Jojoba oil, then combined with 0.25 oz. melted beeswax. The resulting substance was applied to each infected toenail after application of 2% sodium chlorite solution.

Composition 15. Composition 14 was applied immediately before application of 1% Tolnaftate.

Composition 16. (T+MA+GS+Beeswax) A solution containing 3.66 g menthol crystals was liquefied overnight in 100 ml of grape seed oil, 3.66 g thymol crystals was added then heated until liquefied. Next, 0.25 oz. of melted beeswax was added to the composition. The resulting substance was applied to infected skin and toenails after application of 2% sodium chlorite solution.

Composition 17. A solution containing Kerasal containing urea was applied to nail after one drop of 2% sodium chlorite once daily for seven days. Nail degraded severely by horizontal delamination. Treatment was stopped. Composition 2 and 1% terbinafine were combined and was applied to infected skin and toenails after application of 2% sodium chlorite solution. Nails improved aesthetically particularly in regard to smoothing the appearance of horizontal delamination.

Treatments described herein promote healthy disease-free nail growth but do not rely on nail growth as a measure of positive results. Even portions of the nail that are discolored and distorted by the presence of fungus return to an aesthetically pleasing normal healthy color and texture when the treatments described herein are applied.

Table 21 provides an evaluation of the compositions in terms of fungus quality, skin roughness, healthy nail appearance, and irritation of nail and skin.

TABLE 21
Results of Onychcomycosis

| | Fungus | Skin | Nail | |
|---|---|---|---|---|
| | Quality | Roughness | Appearance | Irritation |
| Composition 1 | 2 | 2 | 1 | 1 |
| Composition 2 | 2 | 1 | 1 | 1 |
| Composition 3 | 2 | 1 | 1 | 1 |
| Composition 4 | 2 | 1 | 1 | 1 |
| Composition 5 | 2 | 2 | 2 | 1 |
| Composition 6 | 1 | 1 | 1 | 1 |
| Composition 6a | 1 | 1 | 1 | 1 |
| Composition 7 | 1 | 1 | 1 | 1 |
| Composition 8 | 1 | 1 | 1 | 1 |
| Composition 9 | 1 | 1 | 1 | 1 |
| Composition 9 | 1-2 | 1-2 | 1-2 | 1 |
| Composition 10 | 1 | 1 | 1 | 1 |
| Composition 11 | 1 | 1 | 1 | 1 |
| Composition 12 | 1 | 2-1 | 1 | 1 |
| Composition 13 | 1-2 | 2-1 | 1-2 | 1 |
| Composition 14 | 2-1 | 2-1 | 2-1 | 1 |
| Composition 15 | 1-2 | 1-2 | 1-2 | 1 |
| Composition 16 | 2-1 | 2-1 | 1-2 | 1 |

### Example 10

### Treatment of Dog Wound

Dog was occasionally limping and favoring his rear leg. A 2-inch diameter area of raw red moist skin was observed where hair had fallen out. Upon close inspection a 1½ inch suppurating cut was observed which created a dark line leading to a 1 cm×2 cm section of blackened skin. Dog had been topically treated with Triamcinolone Acetonide and bandaged previously to no effect.

Composition consisting of 2% sodium chlorite solution mixed in hand with 3.8% thymol, 3.8% menthol and 3.8% menthyl acetate in 33% jojoba oil and 66% grapeseed oil and beeswax as thickener was applied to wound 3 times at twelve hour intervals. The ratio was 1:1. At the second and third interval Neosporin™ was mixed in hand with other compositions in a 1:1:1 ratio. Dog was not observed to lick wound. Dog calmly accepted application of treatment evidencing no pain or irritation.

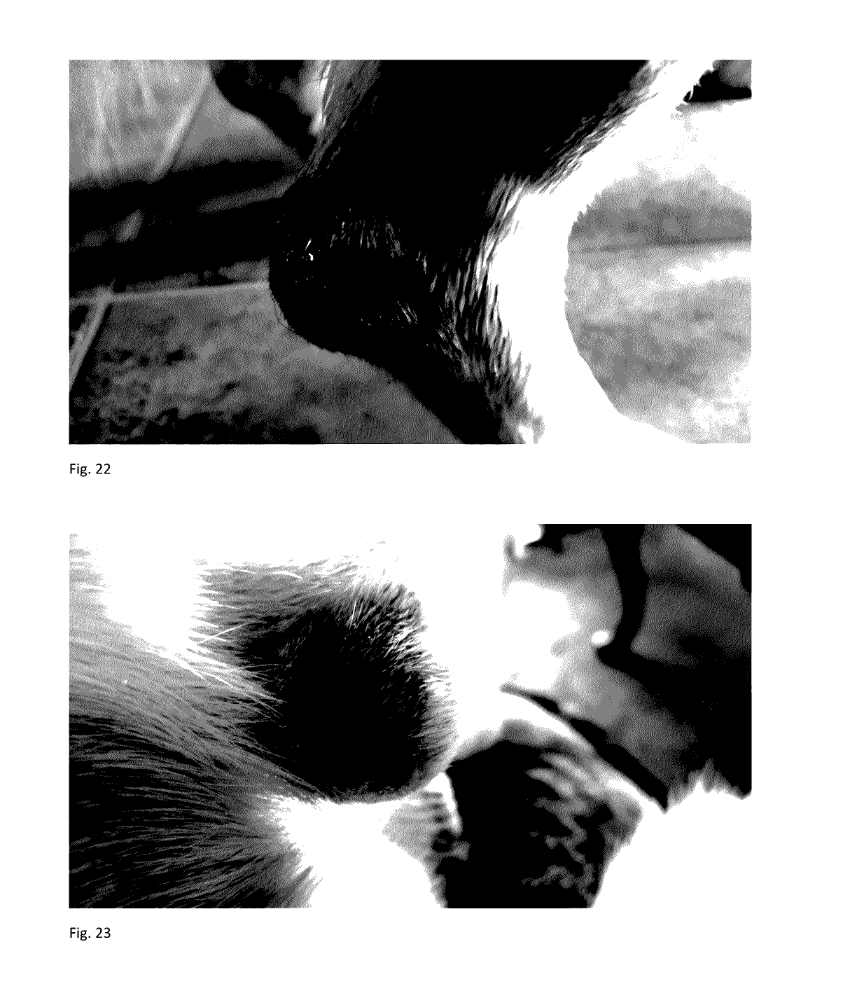

Results: Wound is notably better within 30 hours. Blackened and red patches of skin are now pink except for a remaining ½ cm patch. Dog is no longer favoring wound or limping. FIG. 22 illustrates the wound prior to treatment. FIG. 23 illustrates the wound after three applications, 32 hours after the initial treatment.

### Example 11

### Onychomycosis

Subject Background: 56-year-old female was diagnosed with onychomycosis by her dermatologist. She has had symptoms for the last ten years. She tried numerous over the counter products with little discernible improvement.

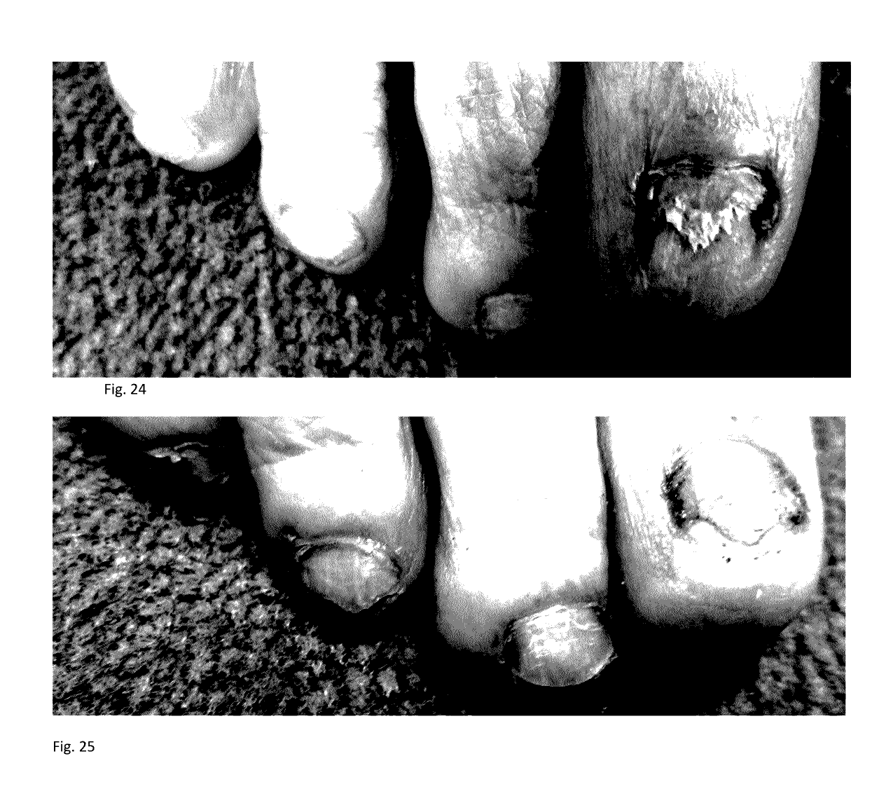

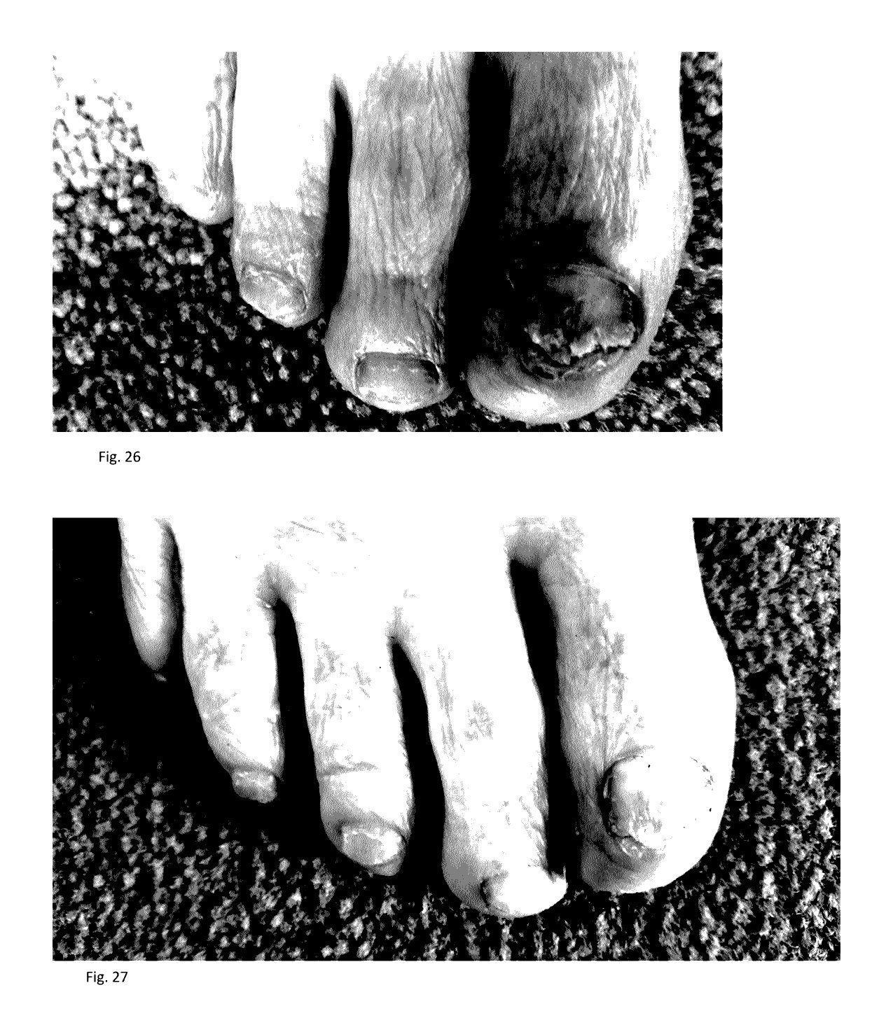

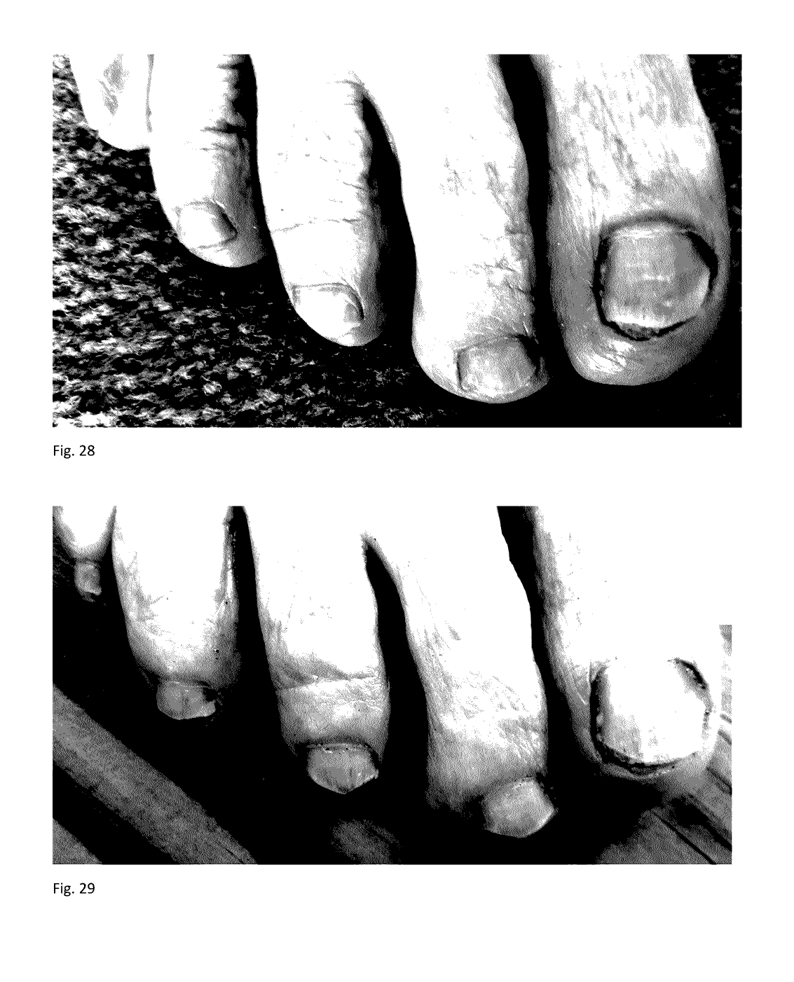

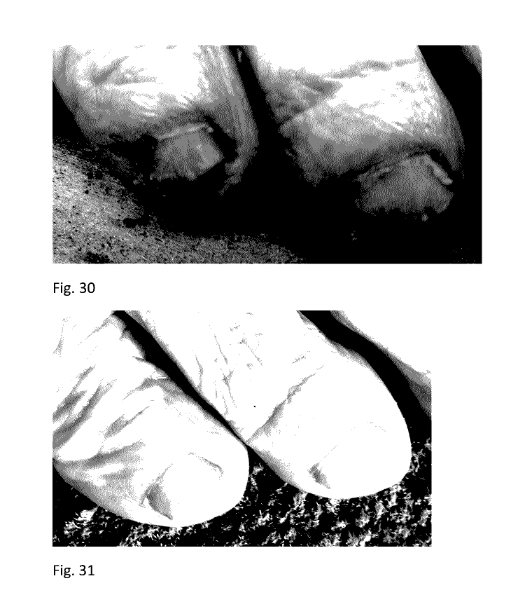

Right foot: FIG. 24 illustrates the baseline right foot. FIG. 25 illustrates the right food after 42 days of applying the Composition 14, a solution containing 3.736 g thymol crystals and 3.736 g menthol crystals and 3.736 g of menthyl acetate liquefied in 66 ml of grape seed oil and 33 ml of Jojoba oil, then combined with 0.25 oz. melted beeswax. The resulting substance was applied to each infected toenail after application of 2% sodium chlorite solution. Composition 14 was applied once daily with no pharmaceutical antifungals (about two treatments were missed). FIG. 26 illustrates the right foot after 77 days of using Composition 14 once daily with no pharmaceutical antifungals. FIG. 27 illustrates treatment after 91 days. FIG. 28 illustrates the right foot after six months of treating the foot with Composition 14 and 1% Clotrimazole once daily (approximately four treatments were missed). FIG. 29 shows the right foot after six months and 25 days of treatment of Composition 14 and 1% Clotrimazole once daily. FIG. 30 illustrates the baseline of toes 3 and 4 prior to Composition 14/Terbinafine treatment, and FIG. 31 illustrates toes 3 and 4 after one week of treatment with sodium chlorite/terbinafine and Composition 14. A reduction in vertical white striations in toe 4 is noted.

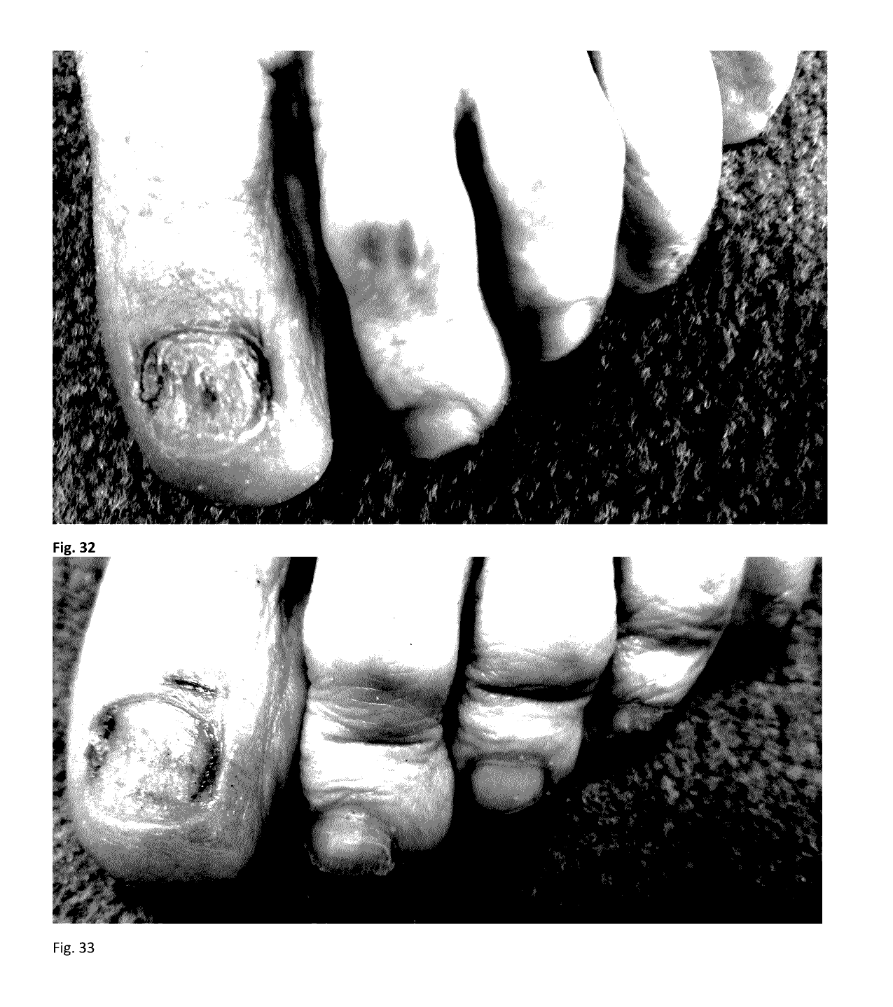

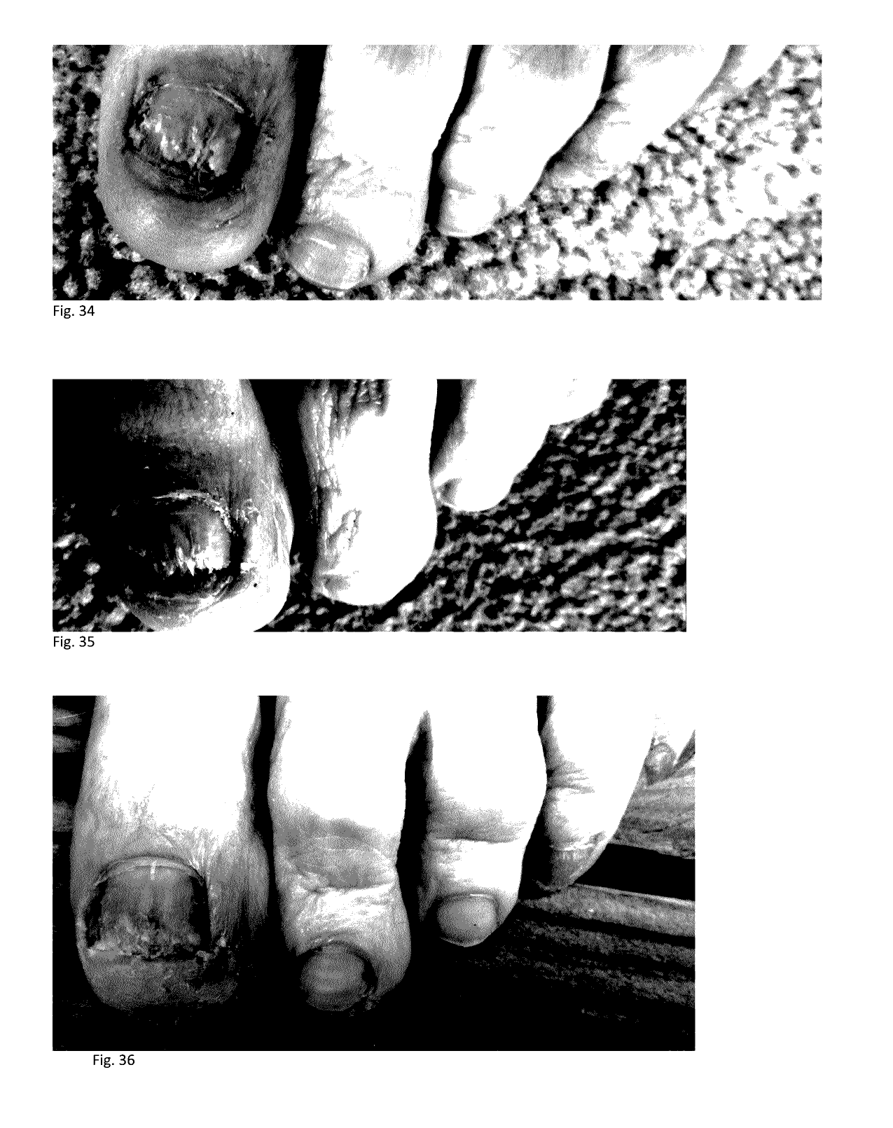

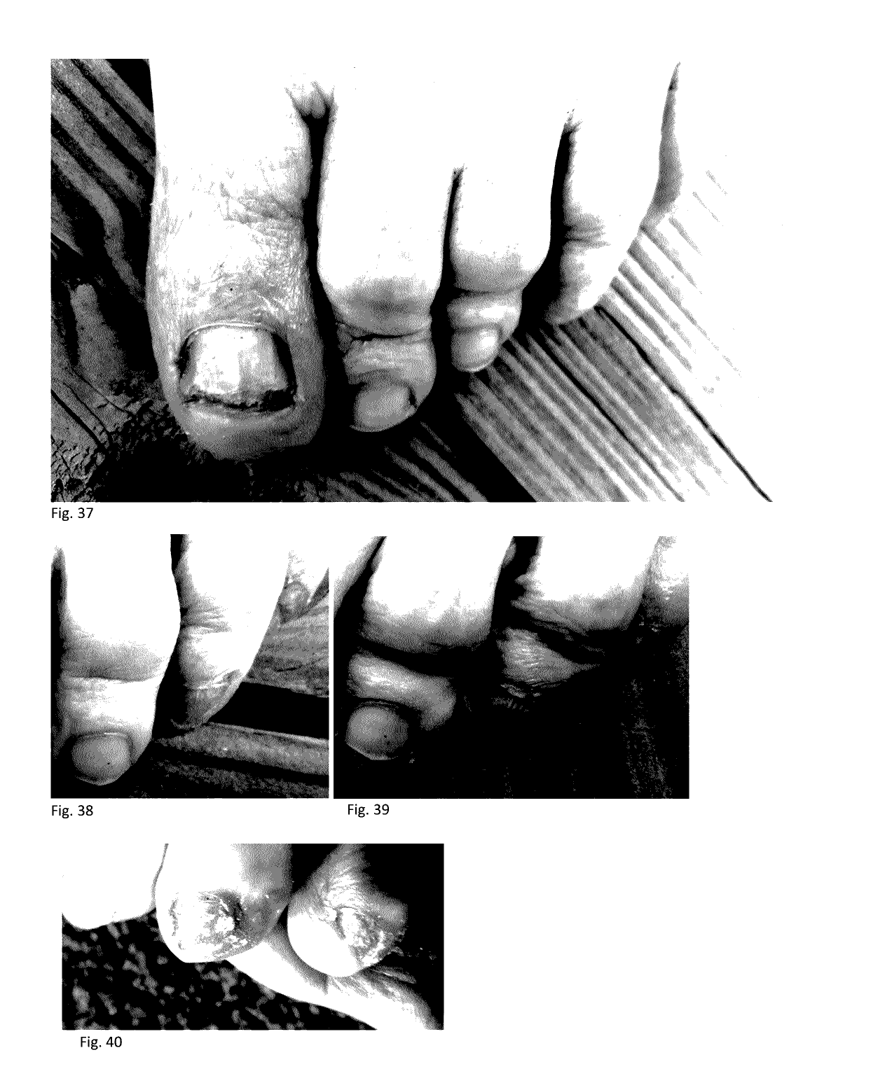

Left foot: FIG. 32 illustrates the baseline left foot. FIG. 33 illustrates the left foot after 42 days of treatment using Composition 14 and 1% Clotrimazole once daily (approximately two treatments were missed). FIGS. 34 and 35 show the left foot after 77 days and 91 days, respectfully, of treatment using Composition 14 and 1% Clotrimazole once daily. FIG. 36 illustrates the left foot after six months of treatment. FIG. 37 show the left foot after six months and 25 days of treatment using Composition 14 and 1% Clotrimazole once daily (missing four treatments). FIGS. 38 and 39 show the baseline for toes 3, 4, and 5, and FIG. 40 illustrates toes 3, 4, and 5 after one week of treatment with Kerasel and sodium chlorite on toe 5 and Kerasal alone on toe 4.

Though natural oils are preferred, the lipid phase of the topical compositions can be chosen from: mineral oils and mineral waxes; oils such as triglycerides of caprinic acid or caprylic acid and castor oil; oils or waxes and other natural or synthetic oils, esters of fatty acids with alcohols e.g. isopropanol, propylene glycol, glycerin or esters of fatty alcohols with carboxylic acids or fatty acids; alkylbenzoates; and/or silicone oils such as dimethylpolysiloxane, diethylpolysiloxane, diphenylpolysiloxane, cyclomethicones and mixtures thereof.

Half synthetic oils are acceptable, (e.g. cocoglyceride, olive oil, sun flower oil, soybean oil, peanut oil, rape seed oil, sweet almond oil, palm oil, coconut oil, castor oil, hydrogenated castor oil, wheat oil, grape seed oil, macadamia nut oil and others). Acceptable oils include but are not limited to apolar oils such as linear and/or branched hydrocarbons and waxes e.g. mineral oils, vaseline (petrolatum); paraffins, squalane and squalene, polyolefines, hydrogenated polyisobutenes and isohexadecanes, dialkyl ethers such as dicaprylylether; linear or cyclic silicone oils such as cyclomethicones (octamethylcyclotetrasiloxane; Dimethicones including but not limited to PPG-2 and cetyldimethicone, hexamethylcyclotrisiloxane, polydimethylsiloxane, montoperones, poly(methylphenylsiloxane) and mixtures thereof.

Other fatty components suitable for use in the topical compositions of the present invention include polar oils such as lecithines and fatty acid triglycerides, namely triglycerol esters of saturated and/or unsaturated, straight or branched carboxylic acid. A non-limiting list of other fatty components that may be incorporated in topical compositions of the present invention are isoeikosane; neopentylglykoldiheptanoate; propyleneglykoldicaprylate/dicaprate; caprylic/capric/diglycerylsuccinate; butyleneglycol caprylat/caprat; C.sub.12-13-alkyllactate; di-C.sub.12-13 alkyltartrate; triisostearin; dipentaerythrityl hexacaprylat/hexacaprate; propylenglycolmonoisostearate; tricaprylin; dimethylisosorbid, the use of mixtures C.sub.12-15-alkylbenzoate and 2-ethylhexylisostearate, mixtures C.sub.12-15-alkylbenzoate and isotridecylisononanoate as well as mixtures of C.sub.12-15-alkylbenzoate, 2-ethylhexylisostearate and isotridecylisononanoate.

The oily phase of the compositions of the present invention can also contain natural vegetable or animal waxes such as bee wax, china wax, bumblebee wax and other waxes of insects as well as shea butter and cocoa butter.

Fatty substances which can be incorporated in the oil phase of the emulsion. Microemulsion, oleo gel, hydrodispersion or lipodispersion of the present invention can be chosen from esters of saturated and/or unsaturated, linear or branched alkyl carboxylic acids. Such esters include but are not limited to Isopropol Palmitate, octylpalmitate, octylcocoate, octylisostearate, octyldodecylmyristate, cetearylisononanoate, isopropylmyristate, isopropylpalmitate, isopropylstearate, isopropyloleate, n-butylstearate, n-hexyllaureate, n-decyloleat, isooctylstearate, isononylstearate, isononylisononanoate, 2-ethyl hexylpalmitate, 2-ethylhexyllaurate, 2-hexyldecylstearate, 2-octyldodecylpalmitate, stearylheptanoate, oleyloleate, oleylerucate, erucyloleate, erucylerucate, tridecylstearate, tridecyltrimellitate, as well as synthetic, half-synthetic or natural mixtures of such esters, for example jojoba oil.

Compositions in accordance with the invention can be in the form of a liquid, a lotion, a thickened lotion, a gel, a cream, a milk, an ointment, a paste, a powder, a tinted nail cosmetic make-up, a nail polish with brush applicator, or a solid tube stick and can be optionally be packaged as an aerosol and can be provided in the form of a mousse such as a aerosol mousse, a foam or a spray foams, sprays, sticks, a gel, a plaster, a powder, a cleanser, a soap or aerosols or wipes.

The compositions of the invention can also contain usual cosmetic or pharmaceutical additives, such as preservatives/antioxidants, fatty substances/oils, water, organic solvents, silicones, thickeners, softeners, emulsifiers, sunscreens, antifoaming agents, moisturizers, fragrances, surfactants, fillers, sequestering agents, anionic, cationic, nonionic or amphoteric polymers or mixtures thereof, propellants, acidifying or basifying agents, dyes, colorants, pigments or nanopigments, or any other ingredients usually formulated into cosmetics or medicaments.

A moisturizing agent may be incorporated into a topical composition of the present invention. Suitable humectants can be incorporated into a topical composition of the present invention such as glycerin, polypropylene glycol, polyethylene glycol, lactic acid, pyrrolidon carboxylic acid, urea, phopholipids, collagen, elastin, ceramides, lecithin sorbitol, PEG-4, and mixtures thereof. Additional suitable moisturizers are polymeric moisturizers of the family of water soluble and/or swellable/and/or with water gelating polysaccharides such as hyaluronic acid, chitosan and/or a fucose rich polysaccharide which is e.g. available as Fucogel® 1000 (CAS-Nr. 178463-23-5) by SOLABIA S.

The topical compositions of the present invention can contain the usual cosmetic or pharmaceutical additives such as but not limited to alcohols, including ethanol and/or isopropanol, cetanol, stearyl, low diols or polyols and their ethers, such as but not limited to polyoxyethylene cetyl ether; propyleneglycol, Dipropylene Glycol Dibenzoate, dicarylate dicaptrate, dicarylate dicaptrate, glycerin, ethyleneglycol, ethyleneglycol monoethyl- or monobutylether, propyleneglycol monomethyl- or -monoethyl- or -monobutylether, diethyleneglycol monomethyl- or monoethylether and analogue products, polymers, Capric Triglycerides, Isopropyl Palminate, Polyquaterternium 37, Dicaprylate Dicaprate, Phemerol chloride, Cetrimonium Chloride, (attracts proteins), Allantoin, Didecyldimonium Chloride, Quaternium-15, foam stabilisators; electrolytes and especially one or more thickeners.

A non-limiting list of thickeners that may be used in formulations of the present invention to assist in making the consistency of a product suitable include cocamidopropyl betaine, cocamidopropylamine oxide, carbomer, siliciumdioxide, magnesium and/or aluminium silicates, beeswax, stearic acid, stearyl alcohol polysaccharides and their derivatives such as xanthan gum, hydroxypropyl cellulose, polyacrylamides, acrylate crosspolymers preferably a carbomer, such as Carbopole® of type 980, 981, 1382, 2984, 5984 alone or mixtures thereof.

Suitable neutralizing agents which may be included in the composition of the present invention to neutralize components such as e.g. an emulsifier or a foam builder/stabilizer.

Compositions in accordance with the invention can be in the form of a liquid, a lotion, a thickened lotion, a gel, a cream, a milk, an ointment, a paste, a powder, a make-up, or a solid tube stick and can optionally be packaged as an aerosol and can be provided in the form of a mousse such as a aerosol mousse, a foam or a spray foams, sprays, sticks, a gel, a plaster, a powder, a cleanser, a nail polish, a soap or aerosols or wipes.

Emulsifiers such as but not limited to sorbitan monostearate, cocamidopropyl betaine (organic from coconut oil) and solubizers may be used in the present invention.

The compositions of the invention can also contain usual cosmetic or pharmaceutical additives, such as preservatives/antioxidants, fatty substances/oils, water, organic solvents, silicones, thickeners, softeners, emulsifiers, sunscreens, antifoaming agents, moisturizers, fragrances, surfactants, fillers, sequestering agents, anionic, cationic, nonionic or amphoteric polymers or mixtures thereof, propellants, acidifying or basifying agents, dyes, colorants, pigments or nanopigments, or any other ingredients usually formulated into cosmetics including nail polish or medicaments.

The compositions of the present invention may also include the usual nail polish additives such as but not limited to Ethyl acetate, Butyl acetate, Nitrocellulose, Tribenzoin, Propyl acetate, Acetyl Tributyl, Citrate, Stearalkonium Bentoinite, silica, HDI/Trimethhylol Hexyllactone, Panthenol, Ascorbyl pamitate, silk powder, Acyrlates copolymer, Tetrabutyl phenyl hydroxybenzoate, Alumina, acrylate copolymer/styrene-acrylate copolymer propylene glycol, n-butyl ether and colorants. A water based polish is preferred.

Though Vitamin E Acetate is preferred, other antioxidants such as but not limited to Tetrabutyl phenyl hydroxybenzoate may be used according to this invention.

Preservatives such as but not limited to, propylparaben, Sodium propyl p-hydroxybenzoate, butylated hydroxytoulene msds and sigma and/or butylated hydroxyanisole msds, Methylparaben, Butyl Paraben and/or Propyl paraben may be used. Tennox may be used.

Though sodium chlorite is exemplified, any nail penetrator can be used according to this invention such as but not limited to DMSO.

1. A composition used to treat a dermatological condition comprising a plant-based antifungal and a metal chlorite salt.
